(19) United States
(12) Patent Application Publication
(10) Pub. No.: US 2024/0081377 A1
(43) Pub. Date: Mar. 14, 2024

(54) USING ORGANOIDS AND/OR SPHEROIDS TO CULTIVATE MEAT

(71) Applicant: Orbillion Bio, Inc., San Francisco, CA (US)

(72) Inventors: Patricia BUBNER, Fremont, CA (US); Laura DALEY, Waterloo, IL (US); Gabriel LEVESQUE-TREMBLAY, Los Angeles, CA (US)

(21) Appl. No.: 18/249,367

(22) PCT Filed: Oct. 19, 2021
(86) PCT No.: PCT/US2021/055556

§ 371 Date: Apr. 17, 2023

Related U.S. Application Data
(60) Provisional application No. 63/093,973, filed on Oct. 20, 2020.
(60) Provisional application No. 63/147,121, filed on Feb. 8, 2021.

(51) Int. Cl. A23L 13/00; C12N 5/077; C12N 5/00

(52) U.S. Cl. CPC A23L 13/00; C12N 5/0658; C12N 5/0062; C12N 2501/105; C12N 2513/00; C12N 2501/113; C12N 2501/12

(57) ABSTRACT

A method is described herein. The method includes: acquiring cells from a non-human animal source such as a tissue biopsy, stem cells, precursor cells, embryonic cells, bone marrow or any combination thereof after identification of immortalized cell lines, improvement of cell lines, or a spontaneous immortalization event; expanding cells from the non-human animal source in a plate or microfluidics chip to facilitate a formation of spheroids or organoids in a range of approximately 10 μm to approximately 10 mm; harvesting the spheroids to initiate further propagation in adherent or suspension cultures; and/or seeding a bioreactor with the adherent or suspension cultures for scale-up of cultivated meat production. The method also includes screening the cells, spheroids, or organoids. The method also includes varying a composition of the spheroids to modify a density of the spheroids and/or properties of a meat product.

### CROSS-REFERENCE

This application claims the benefit of U.S. Patent Application No. 63/093,973 filed Oct. 20, 2020, and U.S. Patent Application No. 63/147,121, filed Feb. 8, 2021, which are hereby incorporated by reference in their entirety.

### BACKGROUND

Current methods for global meat production require substantial amounts of land, food, and other resources to raise traditional domesticated animals for consumption. The cultivated meat, or cellular agriculture, field provides an alternative to this approach and includes the harvesting and scale-up of species-specific cells grown in vitro, thereby alleviating the number and cost of animals raised on rapidly declining land dedicated to animal husbandry to sustainably feed the growing population around the world.

Standard practices for cellular scale-up in the cultivated meat field consist of expanding animal cell lines in planar two-dimensional (2D) culture systems, which are then transferred to bioreactors with or without scaffolds for final scale-up of meat production. Cell densities of mono-layer cultivation systems are directly related to the surface area of the plasticware they are propagated within and cease to expand further once cells are contact inhibited. Moreover, cells must then be detached mechanically or enzymatically prior to harvesting.

Alternatively, cells grown three-dimensionally (3D) interact with their environment, which includes other cells and matrices in 3D, creating a more physiological relevant structure that can be propagated for scale-up in a bioreactor in a more efficient and economical manner. These 3D structures, or spheroids, can then be harvested by centrifugation, bypassing the time and costs associated with mechanical or enzymatical harvesting of monolayer systems.

Examples of related art include:

U.S. Published Patent Application No. 2020/0140821 A1 describes systems and methods for producing cell cultured food products. The cultured food products include sushi-grade fish meat, fish surimi, foie gras, and other food types. Various cell types are utilized to produce the food products and can include muscle, fat, and/or liver cells. The cultured food products are grown in pathogen-free culture conditions without exposure to toxins and other undesirable chemicals.

WO 2015/066377 A1 relates to methods for enhancing cultured meat production, such as livestock-autonomous meat production. In certain aspects, the meat is any metazoan tissue or cell-derived comestible product intended for use as a comestible food or nutritional component by humans, companion animals, domesticated or captive animals whose carcasses are intended for comestible use, service animals, conserved animal species, animals used for experimental purposes, or cell cultures.

U.S. Pat. No. 10,669,524 describes a large-scale cell culture system for producing products without harming animals and a method for making meat products using this cell culture system.

WO 2017/083705 A1 describes improvements to cell culture systems and methods of generating organoids. The system provides a novel spinning bioreactor platform for higher-throughput 3D culturing of stem cells (e.g. human induced pluripotent stem cells (iPSCs) or embryonic stem cells (ESCs)). The system can be widely used as a standard platform to generate stem cell-derived human organoids for any tissue and for high-throughput drug screenings, toxicity testing, and modeling normal human organ development and diseases.

WO 2016/015158 A1 describes cell culture mediums for generating organoids, including tumor organoids.

U.S. Published Patent Application No. 2015/0079238 A1 describes edible microcarriers, including microcarrier beads, microspheres and microsponges, appropriate for use in a bioreactor to culture cells that may be used to form a comestible engineered meat product.

U.S. Pat. No. 8,703,216 B2 describes engineered meat products formed as a plurality of at least partially fused layers, where each layer comprises at least partially fused multicellular bodies comprising non-human myocytes. The engineered meat is comestible. The reference also describes multicellular bodies comprising a plurality of non-human myocytes that are adhered and/or cohered to one another. The multicellular bodies are arranged adjacently on a nutrient-permeable support substrate and maintained in culture to allow the multicellular bodies to at least partially fuse to form a substantially planar layer for use in formation of engineered meat. Mark J. Post, “Cultured Meat from Stem Cells: Challenges and Prospects,” Meat Science, November 2012, Vol. 92, Issue 3, Pages 297-301 describes the generation of bio-artificial muscles from satellite cells for the generation of meat.

Mark J. Post, “An Alternative Animal Protein Source: Cultured Beef,” New York Academy of Sciences, November 2014, Vol. 1328, Pages 29-33 describes the need for technological improvements in the field of culturing beef from bovine skeletal muscle stem cells to create a beef mimic with equivalent taste, texture, and appearance and with the same nutritional value as livestock-produced beef.

Carolyn S. Mattick, “Cellular Agriculture: the Coming Revolution in Food Production,” Bulletin of the Atomic Scientists, 2018, Vol. 74, Issue 1 describes the technology of cellular agriculture.

Mark J. Post, et al., “Scientific, Sustainability and Regulatory Challenges of Cultured Meat,” Nature Food, 2020, Vol. 1, Pages 403-415 describes the challenges with transforming cultured meat into a viable commercial option, covering aspects from cell selection and medium optimization to biomaterials, tissue engineering, regulation and consumer acceptance.

Some systems exist in the art. However, their means of operation are substantially different from the present disclosure, as the other inventions fail to solve all the problems taught by the present disclosure.

### SUMMARY

The present invention and its embodiments relate to methods using organoids and/or spheroids to cultivate meat.

In particular, the present invention describes a method. The method includes numerous process steps, such as: acquiring a tissue biopsy from an animal (e.g., a cow, a pig, a chicken, a fish, a sheep, a bison, a wagyu cattle, a duck, a goose, an elk, a deer, a Berkshire pig, a Kurobuta pig, an Iberian pig, and/or an ostrich). It should be appreciated that the animal may be a mammal, an avian, a crustacean, and/or a fish. The method also includes: isolating, expanding, and seeding cells from the tissue biopsy in a microfluidics chip or a 96-well or similar plate to facilitate a formation of spheroids; harvesting the spheroids to initiate further propagation in adherent or suspension cultures; and seeding a bioreactor with the adherent or suspension cultures for scale-up of cultivated meat production. In some embodiments, the present invention also includes expanding, and seeding cells from the tissue biopsy after identification of immortalized cell lines, improvement of cell lines, or a spontaneous immortalization event. In some embodiments, the present invention also includes expanding, and seeding cells from sources other than a tissue biopsy such as stem cells, precursor cells, embryonic cells, bone marrow or any combination thereof after identification of immortalized cell lines, improvement of cell lines, or a spontaneous immortalization event. In some embodiments, the present invention also includes expanding, and seeding cells from sources other than a tissue biopsy such as stem cells, precursor cells, embryonic cells, bone marrow or any combination thereof.

In examples, the spheroids are in a range of approximately 10 μm to approximately 10 mm. In some examples, the spheroids comprise a singular cell type. In other examples, the spheroids comprise a mixture of cell types (e.g., two or more cell types). In further embodiments, the spheroids comprise scaffolding. However, such scaffolding is not necessary in other embodiments. The spheroids may comprise a ratio of muscle to fat, such as a 30:70 ratio, a 50:50 ratio, or other ratios not explicitly listed herein. Moreover, the spheroids may comprise additional cell types, such as endothelial cells, connective tissue, etc., or different cell types at other ratios not explicitly listed.

It should be appreciated that the method may also include: varying a composition of the spheroids to modify a density of the spheroids and/or properties of a meat product. Such properties may include: a texture of the product, a taste of the product, and/or a mouthfeel of the product. Additionally, spheroids may be cultured with cells grown in in monolayers or with the conditioned media from cells grown in monolayers to influence important aspects of the product such as texture, taste or mouthfeel.

In general, the present invention succeeds in conferring the following benefits and objectives.

It is an object of the present invention to provide methods to improve the industrial scale-up and culture process using discrete sizes of high density 3D spheroids for upstream processing.

It is an object of the present invention to provide methods to improve the industrial scale-up and culture process using high-throughput screening to tailor media and/or use different concentrations of growth factor treatments or other treatments in a species and cell type specific manner. This can comprise different cell types, such as embryonic stem cells, iPSCs, muscle cells, fat cells, endothelial cells, connective cells, fibroblasts or others.

It is an object of the present invention to provide methods to improve the industrial scale-up and culture process using high-throughput screening for co-culture conditions of animal cell lines used for cultivated meat and seafood products and consumables.

It is an object of the present invention to provide methods to improve the industrial scale-up and culture process using high-throughput screening for multi-lineage differentiation of animal cells used for cultivated meat and seafood products and consumables.

It is an object of the present invention to provide methods to improve the industrial scale-up and culture process using high-throughput selection for suspension adapted cells of animal cell lines used for cultivated meat and seafood products and consumables.

It is an object of the present invention to provide methods to improve the industrial scale-up and culture process using high-throughput screening of metabolically active spheroids used for cultivated meat and seafood products and consumables.

It is an object of the present invention to provide methods to improve the industrial scale-up and culture process in the field of cultivated meat production.

Described herein in one aspect, is a method of acquiring spheroids for a cultivated meat product comprising: (a) acquiring muscle cells or muscle cell precursors from a non-human animal source; (b) expanding the muscle cells or the muscle cell precursors from the non-human animal source in the presence of an effective concentration of insulin-like growth factor (IGF) in a culture vessel, on a plate, or on a microfluidics chip to facilitate formation of muscle spheroids or organoids; (c) harvesting the spheroids or organoids when the average diameter of the muscle spheroids or organoids is about 100 micrometers or greater; (d) initiating further propagation in adherent or suspension cultures; and optionally seeding a bioreactor with the adherent or suspension cultures for the cultivated meat production. In certain embodiments, acquiring muscle cells or muscle cell precursors from a non-human animal source comprises acquiring cells from a tissue biopsy, an immortalized cell line, blood, stem cells, precursor cells, embryonic cells, bone marrow, or any combination thereof. In certain embodiments, the method includes screening cells, spheroids, or organoids for metabolic activity. In certain embodiments, the method further comprises harvesting the spheroids or organoids when the average diameter of the spheroids are from about 100 micrometers to about 1000 micrometers. In certain embodiments, the muscle spheroids or organoids comprise a single cell type. In certain embodiments, the muscle spheroids or organoids comprise a mixture of two or more cell types. In certain embodiments, the muscle spheroids or organoids further comprise embryonic stem cells, induced pluripotent stem cells, satellite cells, mesenchymal stem cells, and/or hematopoietic stem cells. In certain embodiments, the muscle spheroids or organoids comprise scaffolding. In certain embodiments, the muscle spheroids or organoids do not comprise scaffolding. In certain embodiments, the non-human animal is selected from the group consisting of: a cow, a pig, a chicken, a fish, a sheep, a bison, a duck, a goose, an elk, a deer, a Berkshire pig, a Kurobuta pig, an Iberian pig, and an ostrich. In certain embodiments, the muscle spheroids or organoids are cultured in a heterologous extracellular matrix. In certain embodiments, the muscle spheroids or organoids are cultured in a heterologous extracellular matrix comprising from about 5% to about 15% or from about 6% to about 14% gelatinous protein mixture from heterologous cells. In certain embodiments, the muscle spheroids or organoids are cultured in a heterologous extracellular matrix comprising a hydrogel or laminin. In certain embodiments, the method further comprises expanding the muscle cells or the muscle cell precursors from the non-human animal source by treating the spheroids or organoids with a growth factor selected from: fibroblast growth factor (FGF), hepatocyte growth factor (HGF), and a combination thereof. In certain embodiments, the method further comprises expanding the muscle cells or the muscle cell precursors from the non-human animal source by treating the spheroids or organoids with fibroblast growth factor (FGF) and hepatocyte growth factor (HGF). In certain embodiments, the spheroids or organoids are treated with about 1 to about 20 ng/mL. fibroblast growth factor (FGF). In certain embodiments, the spheroids or organoids are treated with about 5 to about 100 ng/mL hepatocyte growth factor (HGF). In certain embodiments, the spheroids or organoids are treated with about 1 to about 50 ng/mL insulin-like growth factor (IGF). In certain embodiments, the spheroids or organoids are treated with about 5 to about 20 ng/mL insulin-like growth factor (IGF). In certain embodiments, the muscle spheroids or organoids are treated with at least about 5 ng/mL of insulin-like growth factor (IGF). In certain embodiments, harvesting the muscle spheroids or organoids is performed at least 2 days after expanding the muscles cells or muscle precursors from the non-human animal source in the presence of an effective concentration of insulin-like growth factor (IGF). In certain embodiments, harvesting the muscle spheroids or organoids is performed up to 5 days after expanding the muscles cells or muscle precursors from the non-human animal source in the presence of an effective concentration of insulin-like growth factor (IGF). In certain embodiments, harvesting the muscle spheroids or organoids is performed at days 3 or 4 after expanding the muscles cells or muscle precursors from the non-human animal source in the presence of an effective concentration of insulin-like growth factor (IGF). In certain embodiments, the culture vessel, the plate or the microfluidics chip comprise one or more recesses possessing an inverted dome shape and a diameter from about 100 micrometers to about 1,000 micrometers.

Described herein in another aspect is a composition comprising a first type of spheroid comprising a first plurality of non-human animal cells and a second type of spheroid comprising a second plurality of non-human animal cells, wherein the second type of spheroid is different from the first type of spheroid; wherein the first type of spheroid comprises muscle cells or muscle cell precursors from a non-human animal, wherein the diameter of the first and/or second type of spheroid is at least about 100 micrometers or greater, wherein the composition optionally further comprises a sterile medium free of animal serum. In certain embodiments, the first type of spheroid and/or the second type of spheroid comprise two or more cell types. In certain embodiments, the second type of spheroid comprising a second plurality of non-human animal cells, comprises cells of a different tissue type from the plurality of non-human animal cells or cell precursors of the first type of spheroid. In certain embodiments, the first and second type of spheroids form a three-dimensional structure wherein about 50% to about 90% of the first or second spheroids are not in contact with an exogenous support or scaffold. In certain embodiments, the first and second type of spheroids form a three-dimensional structure wherein about 1% to about 49% of the first or second spheroids are not in contact with an exogenous support or scaffold. In certain embodiments, the first type of spheroid has an average diameter that is different from an average diameter of the second spheroid. In certain embodiments, the composition further comprises a third type of spheroid comprising a third plurality of cells, wherein the third plurality of cells is derived from a different tissue type from the first or second spheroid, optionally wherein the third spheroid has an average diameter that is different from the average diameter of the first or second type of spheroid. In certain embodiments, the second plurality of non-human animal cells is selected from a group consisting of muscle cells, connective tissue cells, fat cells, chondrocytes, blood cells, and combinations thereof. In certain embodiments, the second plurality of non-human animal cells is selected from a group consisting of muscle cell precursors, connective tissue cell precursors, fat cell precursors, chondrocyte precursors, blood cell precursors, and combinations thereof. In certain embodiments, the third plurality of non-human animal cells is selected from the group consisting of muscle cells, connective tissue cells, fat cells, chondrocytes, blood cells, and combinations thereof. In certain embodiments, the third plurality of non-human animal cells is selected from the group consisting of muscle cell precursors, connective tissue cell precursors, fat cell precursors, chondrocyte precursors, blood cell precursors, and combinations thereof. In certain embodiments, the three-dimensional structure comprises a surface area of 64 square centimeters to 225 square centimeters. In certain embodiments, the three-dimensional structure comprises a density of 0.3 grams per cubic centimeter to 1.8 grams per cubic centimeter. In certain embodiments, the first type of spheroids, the second type of spheroids, and/or the third type of spheroids have a roundness of 0.1-2. In certain embodiments, the first type of spheroid, second type of spheroid, and/or third type of spheroid possess a diameter from about 100 micrometers to about 10000 micrometers. In certain embodiments, the composition comprises a predetermined ratio of muscle cells to fat cells, muscle cells to connective tissue cells, or connective tissue cells to fat cells. In certain embodiments, the spheroids comprise a specific ratio of muscle cells to fat cells to connective tissue cells. In certain embodiments, the composition further comprises cartilage. In certain embodiments, the composition comprises a percentage of muscle cells or muscle cell precursors from about 50% to about 95%, about 60% to about 95%, or about 75% to about 95%. In certain embodiments, the composition comprises a percentage of connective tissue cells or connective tissue cell precursors from about 1% to 20%, about 1% to 10%, or about 1% to 5%. In certain embodiments, the composition comprises a percentage of fat tissue cells or fat tissue cell precursors from about 1% to 50%, about 1% to 30%, about 1% to 20%, or about 1% to 10%. In certain embodiments, the first plurality of non-human animal cells are harvested from a tissue biopsy of a non-human animal. In certain embodiments, the non-human animal is selected from the group consisting of: a cow, a pig, a chicken, a fish, a sheep, a bison, a boar, a reptile, an ostrich, a sheep, a goat, a camel, a duck, a goose, an elk, a deer, a Berkshire pig, a Kurobuta pig, an Iberian pig, and a turkey. In certain embodiments, the first plurality of non-human animal cells are cultured on a scaffolding or microfluidic chip to facilitate the formation of spheroids. In certain embodiments, the composition further comprises a heterologous extracellular matrix. In certain embodiments, the composition further comprises from about 5% to about 15% or from about 6% to about 14% gelatinous protein mixture.

### BRIEF DESCRIPTION OF THE DRAWINGS

FIG. 1 depicts a schematic diagram of a workflow for cell isolation, according to at least some embodiments disclosed herein.

FIG. 2A depicts an image of a cross-section of a microfluidics chip, generating different spheroid sizes, according to at least some embodiments disclosed herein.

FIG. 2B depicts a graph showcasing spheroid diameters based on cultivation time, according to at least some embodiments disclosed herein.

FIG. 2C depicts a graph showcasing microtissue area based on cultivation time, according to at least some embodiments disclosed herein.

FIG. 2D depicts an image of a roundness factor regarding each well diameter based on cultivation time, according to at least some embodiments disclosed herein.

FIG. 3 depicts a schematic diagram of a method, according to at least some embodiments disclosed herein.

FIG. 4 depicts (A) a rendering of a microfluidics chip, (B) a platform comprising a microfluidic channel structure and a cover layer, and (C) a workflow of on-chip spheroid generation within 24 hours and in-situ analysis of cellular health under various treatment conditions, according to at least some embodiments disclosed herein.

FIG. 5A-5D depicts (FIG. 5A) a spheroid diameter change of three-dimensional (3D) spheroids of A549 cells, (FIG. 5B) a spheroid diameter change of 3D spheroids of HepG2 cells, (FIG. 5C) a spheroid diameter change of 3D spheroids of CacO-2 cells, and (FIG. 5D) a spheroid diameter change of 3D spheroids of NHDF cells, on days 3 (left box) and 12 (right box) post culture according to at least some embodiments disclosed herein. Densities left to right (in cells per mL) 1.0×10⁵, 2.5×10⁵, 5.0×10⁵, 7.5×10⁵, 1.0×10⁶, and 3.0×10⁶.

FIG. 6 depicts (A) tilting schemes of the system by gravity-driven flow, and (B) and (C) bidirectional fluid profiles of flow velocities (mm/s) during tilting of the microfluidics chip, according to at least some embodiments disclosed herein.

FIG. 7A-7D depicts (FIG. 7A) on-chip monitoring of microtissue penetration of 100 μM, 10 μM and 1 μM Doxorubicin (DOX) in A540 spheroids of different sizes over a cultivation period of four hours, where n=6±SD, (FIG. 7B) micrographs of different-sized A549 spheroids in the microfluidics chip treated with Cisplatin (CIS), Doxorubicin (DOX), and a combination of both (CIS:DOX) for 24 hours to screen drug toxicity, (FIG. 7C) dose response relations of CIS and DOX treated A549 spheroids of different sizes in the microfluidics chip for 24 hours, and (FIG. 7D) a statistical analysis of respective CIS and DOX concentrations, according to at least some embodiments disclosed herein.

FIG. 8 depicts an image of spheroid seeding at day three, according to at least some embodiments disclosed herein.

FIG. 9 depicts an image of spheroid seeding at day twelve, according to at least some embodiments disclosed herein.

FIG. 10 depicts an image of three-days post-harvest, according to at least some embodiments disclosed herein.

FIG. 11 depicts an image of six days post-harvest, according to at least some embodiments disclosed herein.

FIG. 12 depicts an image of sheep muscle tissue, cultured according to the methods described herein.

FIG. 13 depicts a method of obtaining and culturing cells according to the methods described herein.

FIG. 14 depicts an image of sheep muscle spheroids, produced according to the methods described herein.

FIG. 15 depicts an image of embryonic stem cells cultured as spheroids at day zero and day one.

FIG. 16 depicts a graph showcasing DNA proliferation based on cultivation time.

FIG. 17 depicts an image of sheep muscle tissue based on cultivation time.

FIG. 18 depicts graphs depicting metabolic activity of 3D tissue spheroids with diameters of (A) 300 (B) 500 μM (FIG. 18B), (C) 700 μM.

FIG. 19A-19D depicts graphs depicting metabolic activity of 3D tissue spheroids with diameters of (FIG. 19A) 300 μM, (FIG. 19B) 500 μM, (FIG. 19C) 700 μM. (FIG. 19D) shows the results for untreated controls.

FIG. 20A-20B depicts (FIG. 20A) graphs depicting spheroid diameter, (FIG. 20B) and change over time in 300 μM wells.

FIG. 21A-21B depicts (FIG. 21A) graphs depicting spheroid diameter, (FIG. 21B) and change over time in 900 μM wells.

FIG. 22 depicts small organoids formed in an Erlenmeyer shaking flask with flat bottom grown in shaking incubator at various RPM.

### DETAILED DESCRIPTION

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Typically, two-dimensional (2D) cell cultures are used to understand the formation of tissue and organs, as well as diseases in vitro. However, 2D cell culture techniques do not directly replicate the mechanical and biochemical signals present in the body. In 2D techniques, cell-to-surface interactions prevail, rather than cell-to-cell and cell- to extracellular matrix (ECM) interactions that form the basis for normal cell function. Since awareness of the relevance of the cellular microenvironment has increased, three-dimensional (3D) cell culture is gaining popularity. 3D cell cultures facilitate the production of homotypic or heterotypic cell cultures in a spatially relevant manner that mimics the natural microenvironment.

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. Compositions for treating or preventing a given disease can consist essentially of the recited active ingredient, exclude additional active ingredients, but include other non-material components such as excipients, carriers, or diluents. “Consisting of” shall mean excluding more than trace elements of other ingredients and substantial method steps. Embodiments defined by each of these transition terms are within the scope of this disclosure.

As used herein the term “heterologous” in reference to a protein, polypeptide, nucleic acid, or compound refers to a substance that is derived from a different source of the pluralities or spheroids into which the heterologous substance is included. In certain cases, a heterologous substance is purified from a different cell population. In certain cases, a heterologous substance is one that is not naturally made, synthesized, or present in the cells of the spheroids or pluralities recited.

As used herein the term “about” refers to an amount that is near the stated amount by 10%. As used herein the term “a.u.” or “arbitrary unit” refers to a relative unit of measurement to show the ratio of amount of quantities or proportions, and serves to compare multiple measurements performed in similar environment since the ratio between measurement and reference is consistent and dimensionless quantity independent of what actual units are used. In certain embodiments, the ratio is a percentage of the proportions of the control sample.

As used herein the term “non-human animal” refers to live organisms that are not human or not Homo sapiens species. In certain embodiments, the individual is a mammal. In certain embodiments, the mammal is a mouse, rat, rabbit, dog, cat, horse, cow, sheep, pig, goat, llama, alpaca, yak, bison, wagyu cattle, boar, elk, deer, or camel. In certain embodiments, the non-human animal is selected from the group consisting of: a cow, a pig, a chicken, a fish, a bird, a sheep, a bison, a wagyu cattle, a boar, a reptile, an ostrich, a sheep, a goat, a camel, a duck, a goose, an elk, a deer, and a turkey.

As used herein the term “non-human animal cells” refers to cells derived from a non-human animal. Examples of non-human animal cells are those cells that can differentiate into or that are derived from one or more types of tissues including muscle, connective tissue, fat, cartilage, liver, heart, eye, skin, lung, intestine, kidney tissue, bone marrow, umbilical cord, and embryonic tissue. The term “muscle cell” refers to a cells which contribute to skeletal contractile motion that form the skeletal muscle tissues of the body, which may include for example a myocyte. The term “muscle cell precursor” refers to myogenic stem cells such as satellite cells. Muscle cells and muscle cell precursors may be isolated from the body of an animal using for example a tissue biopsy. The term “fat cell” refers to a cell that has differentiated and become specialized in the synthesis and storage of fat. In certain embodiments, a fat cell is a lipocyte or adipocyte. In certain embodiments, a “fat cell precursor” refers to cells that develop into fat cells, such as mesenchymal stem cells. In certain embodiments, differentiated fat cells or precursor mesenchymal stem cells may be isolated from the body of an animal using for example a tissue biopsy. The term “connective tissue cell” refers to any of the cells that secrete or differentiate into cells that secrete extra cellular matrix or that may develop into or are of the specialized connective tissue of the body, including but not limited to, areolar, dense, elastic, reticular blood, bone, cartilage, collagen, or any combination thereof. In certain embodiments, a connective tissue cell comprises the tissue that connects, separates, and supports all other types of tissues in the body. In certain embodiments, a connective tissue cell is a fibroblast. In certain embodiments, a fibroblast is a type of biological cell that synthesizes the extracellular matrix and collagen, and produces the structural framework (stroma) for animal tissues. In certain embodiments, a fibroblast or connective tissue cell may be derived from mesenchymal stem cells and/or mesenchyme. The term “cartilage cell” or “chondrocyte” refers to a cell that has differentiated and become specialized in the synthesis and turnover of a large volume of extra cellular matrix (ECM) components such as collagen, glycoproteins, proteoglycans, and hyaluronan. In certain embodiments, chondrocytes vary according to positioning, such as, for example, articular cartilage, including the deep zone, epiphyseal plates, and tissue boundaries. In certain embodiments, a chondrocyte or cartilage cell may be derived from mesenchymal stem cells. The term “chondrocyte precursor” includes cells that develop into chondrocytes, for example, mesenchymal stem cells.

As used herein the term “marbled” refers to a pattern of intramuscular fat tissue within muscle tissue. In some embodiments, the pattern of intramuscular fat tissue contributes to the meat tenderness, juiciness, texture, flavor, appearance, or any combination thereof. In some embodiments the muscle tissue is lean muscle tissue.

As used herein the term “3D”, “3D formation” or “three-dimensional structure” refers to having three dimensions such as height, weight, and depth (or thickness).

### Organoids and Spheroids

As defined herein, a “spheroid” or “organoid” is a type of 3D cell modeling that can simulate a live cell's environmental conditions as compared to a 2D cell model, specifically with the reactions between cells and the reactions between cells and the matrix. Spheroids are useful in the study of changing physiological characteristics of cells, the difference in the structure of healthy cells and tumor cells, and the changes cells undergo when forming a tumor. Spheroids herein may be referred to as a type of spheroid. Spheroids of different types refer to distinct spheroids that are a compositionally distinct. For example, a first type of spheroid may predominantly comprise muscles cells or muscle cell precursors, while a second type of spheroid may predominantly comprise adipose tissue or adipose tissue precursors. Spheroid types may comprise heterogenous mixtures of different cell types.

A “cellular spheroid”, as used herein, is a 3D cell aggregate in the form of a spheroid or having a spheroid-like form. Cellular spheroids can be formed by eukaryotic cells, and in particular, mammalian cells (e.g. human cells), whereby particularly preferred cells are cells being present in organs and tissues of mammals. These spheroids may comprise one or more type of cells. Spheroids function as a promising model for assessing therapeutic treatments, like chemotherapy, cell- and antibody based immunotherapy, gene therapy and combinatorial therapies. The 3D spheroid model can be used to improve the delivery system for compound penetration and targeting into tissues. The use of different type of cells allows to produce more complex “organoids” or tissue-like structures. Moreover, the spheroids can be loosely aggregated, and thus, represent a miniaturized model of a high density cell culture. Further, there is no requirement that spheroids need to stay in the 3D organization for scale up; in fact, they may dissociate.

In some embodiments, a composition comprises a first type of spheroid comprising a first plurality of non-human animal cells, wherein said first plurality of non-human cells interact by one or more interactions selected form the list consisting of cell-to-cell, cell- to extracellular matrix (ECM) interactions, and a combination thereof, optionally wherein the composition further comprises a sterile medium free of animal serum. In some embodiments, a composition comprises a first type of spheroid comprising a first plurality of the non-human animal cells. In some embodiments, the non-human animal cells interact by one or more interactions selected form the list consisting of cell-to-cell, cell- to extracellular matrix (ECM) interactions, and a combination thereof. In some embodiments, a composition comprises a first type of spheroid comprising a first plurality of non-human animal cells, wherein the spheroid comprises two or more cell types.

In some embodiments, a composition comprises said first type of spheroid comprising a first plurality of non-human animal cells, further comprising a second type of spheroid comprising a second plurality of non-human animal cells, wherein the second plurality of non-human animal cells is of a different tissue type from the plurality of non-human animal cells of the first spheroid. In some embodiments, a composition comprises further comprises a second type of spheroid comprising a second plurality of non-human animal cells, optionally wherein the spheroid comprises two or more cell types.

In some embodiments, a composition compresses a first type of spheroid comprising a first plurality of non-human animal cells, a second type of spheroid comprising a second plurality of non-human animal cells, wherein said cells interact by one or more interactions selected form the list consisting of cell-to-cell, cell- to extracellular matrix (ECM) interactions, and a combination thereof, wherein the second plurality of cells is of a different tissue type from the plurality of non-human animal cells of the first type of spheroid, and optionally wherein the composition further comprises a sterile medium free of animal serum.

In some embodiments, a composition comprises a first type of spheroid comprising a first plurality of non-human animal cells, a second type of spheroid comprising a second plurality of non-human animal cells, wherein said cells interact by one or more interactions selected form the list consisting of cell-to-cell, cell- to extracellular matrix (ECM) interactions, and a combination thereof, and optionally wherein the composition further comprises a sterile medium free of animal serum.

In some embodiments, a composition further comprises a plurality of types of spheroids, wherein the spheroids form a three-dimensional structure wherein about 50% to about 90%, about 50% to about 80%, about 50% to about 70%, about 50% to about 60%, 60% to about 90%, 70% to about 90%, 80% to about 90%, of the plurality of spheroids are not in contact with an exogenous support or scaffold. In some embodiments, a composition wherein the first and second types of spheroids form a three-dimensional structure wherein about 50% to about 90% of the first or second spheroids are not in contact with an exogenous support or scaffold.

In some embodiments, a composition further comprises a plurality of types of spheroids, wherein the spheroids form a three-dimensional structure wherein about 1% to about 49%, about 1% to about 49%, about 1% to about 40%, about 1% to about 30%, about 1% to about 20%, about 1% to about 10%, of the plurality of spheroids are in contact with an exogenous support or scaffold. In some embodiments, a composition wherein the first and second types of spheroids form a three-dimensional structure wherein about 1% to about 49% of the first or second spheroids are in contact with an exogenous support or scaffold. In some embodiments, a composition further comprising multiple types of spheroids, wherein the spheroids form a three-dimensional structure. In some embodiments, a composition further comprising multiple types of spheroids, wherein the spheroids form a three-dimensional structure in contact with an exogenous support or scaffold. In some embodiments, a composition further comprising a third type of spheroid comprises a third plurality of non-human animal cells, wherein the third plurality of non-human animal cells is derived from a different tissue type from the tissue type of the first or second spheroid. In some embodiments, a composition comprising multiple types of spheroids, wherein any two or more of the first type of spheroid, the second type of spheroid, and the third type of spheroid are in physical contact.

In some embodiments, a composition as previously described comprises a plurality of spheroids of two or more types, wherein the first type of spheroid possesses a first average diameter that is different from a second average diameter of the second type of spheroid. In some embodiments, a composition as previously described comprising a plurality of spheroids, wherein the first type of spheroid, the second type of spheroid, and/or the third type of spheroid possesses different average diameters. In some embodiments, a composition as previously described comprising a plurality of spheroids of two or more types, wherein each type of spheroid possesses an average diameter that is different from the average diameter of the other spheroid types. In some embodiments, a composition as previously described comprising a plurality of spheroids, wherein the first type of spheroid, the second type of spheroid, and/or the third type of spheroid possesses a diameter from about 10 to about 10000 micrometers.

In some embodiments, a composition as previously described comprises a plurality of spheroids, wherein the first type of spheroid, the second type of spheroid, and/or the third type of spheroid possesses a diameter from about 10 micrometers to about 10,000 micrometers. In some embodiments, a composition as previously described comprising a plurality of spheroids, wherein the first type of spheroid, the second type of spheroid, and/or the third type of spheroid possesses a diameter from about 10 micrometers to about 100 micrometers, about 10 micrometers to about 500 micrometers, about 10 micrometers to about 1,000 micrometers, about micrometers to about 2,500 micrometers, about 10 micrometers to about 5,000 micrometers, about 10 micrometers to about 7,500 micrometers, about 10 micrometers to about 9,000 micrometers, about 10 micrometers to about 10,000 micrometers, about 100 micrometers to about 500 micrometers, about 100 micrometers to about 1,000 micrometers, about 100 micrometers to about 2,500 micrometers, about 100 micrometers to about 5,000 micrometers, about 100 micrometers to about 7,500 micrometers, about 100 micrometers to about 9,000 micrometers, about 100 micrometers to about 10,000 micrometers, about 500 micrometers to about 1,000 micrometers, about 500 micrometers to about 2,500 micrometers, about 500 micrometers to about 5,000 micrometers, about 500 micrometers to about 7,500 micrometers, about 500 micrometers to about 9,000 micrometers, about 500 micrometers to about 10,000 micrometers, about 1,000 micrometers to about 2,500 micrometers, about 1,000 micrometers to about 5,000 micrometers, about 1,000 micrometers to about 7,500 micrometers, about 1,000 micrometers to about 9,000 micrometers, about 1,000 micrometers to about 10,000 micrometers, about 2,500 micrometers to about 5,000 micrometers, about 2,500 micrometers to about 7,500 micrometers, about 2,500 micrometers to about 9,000 micrometers, about 2,500 micrometers to about 10,000 micrometers, about 5,000 micrometers to about 7,500 micrometers, about 5,000 micrometers to about 9,000 micrometers, about 5,000 micrometers to about 10,000 micrometers, about 7,500 micrometers to about 9,000 micrometers, about 7,500 micrometers to about 10,000 micrometers, or about 9,000 micrometers to about 10,000 micrometers. In some embodiments, a composition as previously described comprises a plurality of spheroids, wherein the first type of spheroid, the second type of spheroid, and/or the third type of spheroid possesses a diameter from about 10 micrometers, about 100 micrometers, about 500 micrometers, about 1,000 micrometers, about 2,500 micrometers, about 5,000 micrometers, about 7,500 micrometers, about 9,000 micrometers, or about 10,000 micrometers. In some embodiments, a composition as previously described comprises a plurality of spheroids, wherein the first type of spheroid, the second type of spheroid, and/or the third type of spheroid possesses a diameter from at least about 10 micrometers, about 100 micrometers, about 500 micrometers, about 1,000 micrometers, about 2,500 micrometers, about 5,000 micrometers, about 7,500 micrometers, or about 9,000 micrometers. In some embodiments, a composition as previously described comprises a plurality of spheroids, wherein the first type of spheroid, the second type of spheroid, and/or the third type of spheroid possesses a diameter from at most about 100 micrometers, about 500 micrometers, about 1,000 micrometers, about 2,500 micrometers, about micrometers, about 7,500 micrometers, about 9,000 micrometers, or about 10,000 micrometers.

The compositions described herein may be assembled from a plurality of different spheroid types. In some embodiments, the first type of spheroid, the second type of spheroid, and/or the third type of spheroid possesses a diameter of about 100 micrometers to about 1,000 micrometers. In some embodiments, the first type of spheroid, the second type of spheroid, and/or the third type of spheroid possesses a diameter of about 100 micrometers to about 200 micrometers, about 100 micrometers to about 300 micrometers, about 100 micrometers to about 400 micrometers, about 100 micrometers to about 500 micrometers, about 100 micrometers to about 600 micrometers, about 100 micrometers to about 700 micrometers, about 100 micrometers to about 800 micrometers, about 100 micrometers to about 900 micrometers, about 100 micrometers to about 1,000 micrometers, about 200 micrometers to about 300 micrometers, about 200 micrometers to about 400 micrometers, about 200 micrometers to about 500 micrometers, about 200 micrometers to about 600 micrometers, about 200 micrometers to about 700 micrometers, about 200 micrometers to about 800 micrometers, about 200 micrometers to about 900 micrometers, about 200 micrometers to about 1,000 micrometers, about 300 micrometers to about 400 micrometers, about 300 micrometers to about 500 micrometers, about 300 micrometers to about 600 micrometers, about 300 micrometers to about 700 micrometers, about 300 micrometers to about 800 micrometers, about 300 micrometers to about 900 micrometers, about 300 micrometers to about 1,000 micrometers, about 400 micrometers to about 500 micrometers, about 400 micrometers to about 600 micrometers, about 400 micrometers to about 700 micrometers, about 400 micrometers to about 800 micrometers, about 400 micrometers to about 900 micrometers, about 400 micrometers to about 1,000 micrometers, about 500 micrometers to about 600 micrometers, about 500 micrometers to about 700 micrometers, about 500 micrometers to about 800 micrometers, about 500 micrometers to about 900 micrometers, about 500 micrometers to about 1,000 micrometers, about 600 micrometers to about 700 micrometers, about 600 micrometers to about 800 micrometers, about 600 micrometers to about 900 micrometers, about 600 micrometers to about 1,000 micrometers, about 700 micrometers to about 800 micrometers, about 700 micrometers to about 900 micrometers, about 700 micrometers to about 1,000 micrometers, about 800 micrometers to about 900 micrometers, about 800 micrometers to about 1,000 micrometers, or about 900 micrometers to about 1,000 micrometers. In some embodiments, the first type of spheroid, the second type of spheroid, and/or the third type of spheroid possesses a diameter of about 100 micrometers, about 200 micrometers, about 300 micrometers, about 400 micrometers, about 500 micrometers, about 600 micrometers, about 700 micrometers, about 800 micrometers, about 900 micrometers, or about 1,000 micrometers. In some embodiments, the first type of spheroid, the second type of spheroid, and/or the third type of spheroid possesses a diameter of at least about 100 micrometers, about 200 micrometers, about 300 micrometers, about 400 micrometers, about 500 micrometers, about 600 micrometers, about 700 micrometers, about 800 micrometers, or about 900 micrometers. In some embodiments, the first type of spheroid, the second type of spheroid, and/or the third type of spheroid possesses a diameter of at most about 200 micrometers, about 300 micrometers, about 400 micrometers, about 500 micrometers, about 600 micrometers, about 700 micrometers, about 800 micrometers, about 900 micrometers, or about 1,000 micrometers.

In some embodiments, a composition as previously described comprises multiple types of spheroids, wherein the first plurality of non-human animal cells, the second plurality of non-human animal cells, and/or the third plurality of non-human animal cells selected from a group consisting of muscle cells, fat cells, connective tissue cells, cartilage cells, liver cells, heart cells, eye cells, kidney cells, skin cells, lung cells, or any combination thereof. In some embodiments, a composition as previously described comprises multiple types of spheroids comprising multiple pluralities of non-human animal cells selected from a group consisting of muscle cells, fat cells, connective tissue cells, cartilage cells, liver cells, heart cells, eye cells, kidney cells, skin cells, lung cells, or any combination thereof. In some embodiments, a composition as previously described comprises multiple types of spheroids comprising multiple pluralities of non-human animal cells selected from a group consisting of muscle cells, connective tissue cells, fat cells, cartilage cells, skin cells, and combinations thereof. In some embodiments, a composition as previously described comprises multiple types of spheroids comprises non-human animal cells selected from a group consisting of muscle tissue, fat tissue, connective tissue, cartilage tissue, liver tissue, heart tissue, eye tissue, kidney tissue, skin tissue, lung tissue, or any combination thereof.

In some embodiments, a composition as previously described comprises multiple types of spheroids, wherein the first type of spheroid, the second type of spheroid, and/or the third type of spheroid possesses a surface area from about 50 cm2 to about 300 cm2. In some embodiments, a composition as previously described comprises multiple types of spheroids, wherein the first type of spheroid, the second type of spheroid, and the third type of spheroid possesses a surface area from about 64 cm2 to about 225 cm2. In some embodiments, a composition as previously described comprises multiple types of spheroids, wherein the first type of spheroid, the second type of spheroid, and/or the third type of spheroid possesses a surface area from about 50 cm2 to about 75 cm2, about 50 cm2 to about 100 cm2, about 50 cm2 to about 125 cm2, about 50 cm2 to about 150 cm2, about 50 cm2 to about 175 cm2, about cm2 to about 200 cm2, about 50 cm2 to about 250 cm2, about 50 cm2 to about 300 cm2, about 75 cm2 to about 100 cm2, about 75 cm2 to about 125 cm2, about 75 cm2 to about 150 cm2, about 75 cm2 to about 175 cm2, about 75 cm2 to about 200 cm2, about 75 cm2 to about 250 cm2, about 75 cm2 to about 300 cm2, about 100 cm2 to about 125 cm2, about 100 cm2 to about 150 cm2, about 100 cm2 to about 175 cm2, about 100 cm2 to about 200 cm2, about 100 cm2 to about 250 cm2, about 100 cm2 to about 300 cm2, about 125 cm2 to about 150 cm2, about 125 cm2 to about 175 cm2, about 125 cm2 to about 200 cm2, about 125 cm2 to about 250 cm2, about 125 cm2 to about 300 cm2, about 150 cm2 to about 175 cm2, about 150 cm2 to about 200 cm2, about 150 cm2 to about 250 cm2, about 150 cm2 to about 300 cm2, about 175 cm2 to about 200 cm2, about 175 cm2 to about 250 cm2, about 175 cm2 to about 300 cm2, about 200 cm2 to about 250 cm2, about 200 cm2 to about 300 cm2, or about 250 cm2 to about 300 cm2. In some embodiments, a composition as previously described comprises multiple types of spheroids, wherein the first type of spheroid, the second type of spheroid, and/or the third type of spheroid possesses a surface area from about 50 cm2, about 75 cm2, about 100 cm2, about 125 cm2, about 150 cm2, about 175 cm2, about 200 cm2, about 250 cm2, or about 300 cm2. In some embodiments, a composition as previously described comprises multiple types of spheroids, wherein the first type of spheroid, the second type of spheroid, and/or the third type of spheroid possesses a surface area from at least about 50 cm2, about 75 cm2, about 100 cm2, about 125 cm2, about 150 cm2, about 175 cm2, about 200 cm2, or about 250 cm2. In some embodiments, a composition as previously described comprises multiple types of spheroids, wherein the first type of spheroid, the second type of spheroid, and/or the third type of spheroid possesses a surface area from at most about 75 cm2, about 100 cm2, about 125 cm2, about 150 cm2, about 175 cm2, about 200 cm2, about 250 cm2, or about 300 cm2.

In some embodiments, a composition as previously described comprises multiple types of spheroids, wherein the first type of spheroid, the second type of spheroid, and/or the third type of spheroid form a three-dimensional structure. In some embodiments, a composition as previously described comprises multiple types of spheroids, wherein the spheroids form a three-dimensional structure. In some embodiments, a composition comprises a three-dimensional structure of a plurality of spheroids, wherein the spheroids are linked, at least partially linked, at least partially fused, or any combination thereof. In some embodiments, a composition comprises a three-dimensional structure of a plurality of spheroids comprises a plurality of cells, or a plurality of tissue such as muscle tissue, fat tissue, connective tissue, cartilage tissue, liver tissue, heart tissue, eye tissue, kidney tissue, endothelial tissue, lung tissue, or any combination thereof.

In some embodiments, a composition as previously described comprises multiple types of spheroids, wherein the spheroids form a three-dimensional structure possesses a density from about 0.1 g/cm3 to about 2.5 g/cm3. In some embodiments, a composition as previously described comprises multiple types of spheroids, wherein the spheroids form a three-dimensional structure possesses a density from about 0.3 g/cm3 to about 1.8 g/cm3. In some embodiments, a composition as previously described comprises multiple types of spheroids, wherein the spheroids form a three-dimensional structure possesses a density from about 0.1 g/cm3 to about 0.3 g/cm3, about 0.1 g/cm3 to about 0.5 g/cm3, about 0.1 g/cm3 to about 1 g/cm3, about 0.1 g/cm3 to about 1.3 g/cm3, about 0.1 g/cm3 to about 1.5 g/cm3, about 0.1 g/cm3 to about 1.8 g/cm3, about 0.1 g/cm3 to about 2 g/cm3, about 0.1 g/cm3 to about 2.5 g/cm3, about 0.3 g/cm3 to about 0.5 g/cm3, about 0.3 g/cm3 to about 1 g/cm3, about 0.3 g/cm3 to about 1.3 g/cm3, about 0.3 g/cm3 to about 1.5 g/cm3, about 0.3 g/cm3 to about 1.8 g/cm3, about 0.3 g/cm3 to about 2 g/cm3, about 0.3 g/cm3 to about 2.5 g/cm3, about 0.5 g/cm3 to about 1 g/cm3, about 0.5 g/cm3 to about 1.3 g/cm3, about 0.5 g/cm3 to about 1.5 g/cm3, about 0.5 g/cm3 to about 1.8 g/cm3, about 0.5 g/cm3 to about 2 g/cm3, about 0.5 g/cm3 to about 2.5 g/cm3, about 1 g/cm3 to about 1.3 g/cm3, about 1 g/cm3 to about 1.5 g/cm3, about 1 g/cm3 to about 1.8 g/cm3, about 1 g/cm3 to about 2 g/cm3, about 1 g/cm3 to about 2.5 g/cm3, about 1.3 g/cm3 to about 1.5 g/cm3, about 1.3 g/cm3 to about 1.8 g/cm3, about 1.3 g/cm3 to about 2 g/cm3, about 1.3 g/cm3 to about 2.5 g/cm3, about 1.5 g/cm3 to about 1.8 g/cm3, about 1.5 g/cm3 to about 2 g/cm3, about 1.5 g/cm3 to about 2.5 g/cm3, about 1.8 g/cm3 to about 2 g/cm3, about 1.8 g/cm3 to about 2.5 g/cm3, or about 2 g/cm3 to about 2.5 g/cm3. In some embodiments, a composition as previously described comprises multiple types of spheroids, wherein the spheroids form a three-dimensional structure possesses a density from about 0.1 g/cm3, about 0.3 g/cm3, about 0.5 g/cm3, about 1 g/cm3, about 1.3 g/cm3, about 1.5 g/cm3, about 1.8 g/cm3, about 2 g/cm3, or about 2.5 g/cm3. In some embodiments, a composition as previously described comprises multiple types of spheroids, wherein the spheroids form a three-dimensional structure possesses a density from at least about 0.1 g/cm3, about 0.3 g/cm3, about g/cm3, about 1 g/cm3, about 1.3 g/cm3, about 1.5 g/cm3, about 1.8 g/cm3, or about 2 g/cm3. In some embodiments, a composition as previously described comprises multiple types of spheroids, wherein the spheroids form a three-dimensional structure possesses a density from at most about 0.3 g/cm3, about 0.5 g/cm3, about 1 g/cm3, about 1.3 g/cm3, about 1.5 g/cm3, about 1.8 g/cm3, about 2 g/cm3, or about 2.5 g/cm3.

In some embodiments, a composition as previously described comprises multiple types of spheroids, wherein the first type of spheroid, the second type of spheroid, and/or the third type of spheroid possesses a roundness from about 0.1 A.U. to about 3 A.U. In some embodiments, a composition as previously described comprises multiple types of spheroids, wherein the first type of spheroid, the second type of spheroid, and/or the third type of spheroid possesses a roundness from about 0.1 A.U. to about 2 A.U. In some embodiments, a composition as previously described comprises multiple types of spheroids, wherein the first type of spheroid, the second type of spheroid, and/or the third type of spheroid possesses a roundness from about 0.1 A.U. to about 0.3 A.U., about 0.1 A.U. to about 0.5 A.U., about 0.1 A.U. to about 1 A.U., about 0.1 A.U. to about 1.3 A.U., about 0.1 A.U. to about 1.5 A.U., about A.U. to about 1.8 A.U., about 0.1 A.U. to about 2 A.U., about 0.1 A.U. to about 3 A.U., about 0.3 A.U. to about 0.5 A.U., about 0.3 A.U. to about 1 A.U., about 0.3 A.U. to about 1.3 A.U., about 0.3 A.U. to about 1.5 A.U., about 0.3 A.U. to about 1.8 A.U., about 0.3 A.U. to about 2 A.U., about 0.3 A.U. to about 3 A.U., about 0.5 A.U. to about 1 A.U., about 0.5 A.U. to about 1.3 A.U., about 0.5 A.U. to about 1.5 A.U., about 0.5 A.U. to about 1.8 A.U., about 0.5 A.U. to about 2 A.U., about 0.5 A.U. to about 3 A.U., about 1 A.U. to about 1.3 A.U., about 1 A.U. to about 1.5 A.U., about 1 A.U. to about 1.8 A.U., about 1 A.U. to about 2 A.U., about 1 A.U. to about 3 A.U., about 1.3 A.U. to about 1.5 A.U., about 1.3 A.U. to about 1.8 A.U., about 1.3 A.U. to about 2 A.U., about 1.3 A.U. to about 3 A.U., about 1.5 A.U. to about 1.8 A.U., about 1.5 A.U. to about 2 A.U., about 1.5 A.U. to about 3 A.U., about 1.8 A.U. to about 2 A.U., about 1.8 A.U. to about 3 A.U., or about 2 A.U. to about 3 A.U. In some embodiments, a composition as previously described comprises multiple types of spheroids, wherein the first type of spheroid, the second type of spheroid, and/or the third type of spheroid possesses a roundness from about 0.1 A.U., about 0.3 A.U., about 0.5 A.U., about 1 A.U., about 1.3 A.U., about 1.5 A.U., about 1.8 A.U., about 2 A.U., or about 3 A.U. In some embodiments, a composition as previously described comprises multiple types of spheroids, wherein the first type of spheroid, the second type of spheroid, and/or the third type of spheroid possesses a roundness from at least about 0.1 A.U., about 0.3 A.U., about 0.5 A.U., about 1 A.U., about 1.3 A.U., about 1.5 A.U., about 1.8 A.U., or about 2 A.U. In some embodiments, a composition as previously described comprises multiple types of spheroids, wherein the first type of spheroid, the second type of spheroid, and/or the third type of spheroid possesses a roundness from at most about 0.3 A.U., about 0.5 A.U., about 1 A.U., about 1.3 A.U., about 1.5 A.U., about 1.8 A.U., about 2 A.U., or about 3 A.U.

In some embodiments, a composition as previously described comprises spheroids with a particular roundness, which can be determined by this formula (4×Area)/(π×Major axis\hat{ }2). This formula can determine roundness of one or more spheroids or cells, for example, using software, such as the Clone Select Imager from Molecular Devices to measure roundness and diameter.

In some embodiments, a composition as previously described comprises a plurality of types of spheroids, wherein the first type of spheroid, the second type of spheroid, and/or the third type of spheroid possess a surface area from about 10 mm2 to about 250 mm2. In some embodiments, a composition as previously described comprises multiple types of spheroids, wherein the first type of spheroid, the second type of spheroid, and/or the third type of spheroid possess a surface area from about 10 to about 40 mm2, about 39 to about 100 mm2, or about 90 to about 240 mm2. In some embodiments, a composition as previously described comprises multiple types of spheroids, wherein the first type of spheroid, the second type of spheroid, and/or the third type of spheroid possess a surface area from about 10 mm2 to about 30 mm2, about 10 mm2 to about 40 mm2, about 10 mm2 to about 50 mm2, about 10 mm2 to about 90 mm2, about 10 mm2 to about 100 mm2, about 10 mm2 to about 150 mm2, about 10 mm2 to about 200 mm2, about 10 mm2 to about 250 mm2, about 30 mm2 to about 40 mm2, about 30 mm2 to about 50 mm2, about 30 mm2 to about 90 mm2, about 30 mm2 to about 100 mm2, about 30 mm2 to about 150 mm2, about 30 mm2 to about 200 mm2, about 30 mm2 to about 250 mm2, about 40 mm2 to about 50 mm2, about 40 mm2 to about 90 mm2, about 40 mm2 to about 100 mm2, about 40 mm2 to about 150 mm2, about 40 mm2 to about 200 mm2, about 40 mm2 to about 250 mm2, about 50 mm2 to about 90 mm2, about 50 mm2 to about 100 mm2, about 50 mm2 to about 150 mm2, about 50 mm2 to about 200 mm2, about 50 mm2 to about 250 mm2, about 90 mm2 to about 100 mm2, about 90 mm2 to about 150 mm2, about 90 mm2 to about 200 mm2, about 90 mm2 to about 250 mm2, about 100 mm2 to about 150 mm2, about 100 mm2 to about 200 mm2, about 100 mm2 to about 250 mm2, about 150 mm2 to about 200 mm2, about 150 mm2 to about 250 mm2, or about 200 mm2 to about 250 mm2. In some embodiments, a composition as previously described comprises multiple types of spheroids, wherein the first type of spheroid, the second type of spheroid, and/or the third type of spheroid possess a surface area from about 10 mm2, about 30 mm2, about 40 mm2, about 50 mm2, about 90 mm2, about 100 mm2, about 150 mm2, about 200 mm2, or about 250 mm2. In some embodiments, a composition as previously described comprises multiple types of spheroids, wherein the first type of spheroid, the second type of spheroid, and/or the third type of spheroid possess a surface area from at least about 10 mm2, about 30 mm2, about 40 mm2, about 50 mm2, about 90 mm2, about 100 mm2, about 150 mm2, or about 200 mm2. In some embodiments, a composition as previously described comprises multiple types of spheroids, wherein the first type of spheroid, the second type of spheroid, and/or the third type of spheroid possess a surface area from at most about 30 mm2, about 40 mm2, about 50 mm2, about 90 mm2, about 100 mm2, about 150 mm2, about 200 mm2, or about 250 mm2.

In some embodiments, a composition as previously described comprises multiple types of spheroids, wherein the first type of spheroid, the second type of spheroid, and/or the third type of spheroid possess a volume from about 0.5 mm3 to about 20 mm3. In some embodiments, a composition as previously described comprises multiple types of spheroids, wherein the first type of spheroid, the second type of spheroid, and/or the third type of spheroid possess a volume from about 1 to about 5 mm3, about 4 to about 10 mm3, or about 9 to about 15 mm3. In some embodiments, a composition as previously described comprises multiple types of spheroids, wherein the first type of spheroid, the second type of spheroid, and/or the third type of spheroid possess a volume from about 0.5 mm3 to about 1 mm3, about 0.5 mm3 to about 4 mm3, about mm3 to about 5 mm3, about 0.5 mm3 to about 9 mm3, about 0.5 mm3 to about 10 mm3, about 0.5 mm3 to about 15 mm3, about 0.5 mm3 to about 20 mm3, about 1 mm3 to about 4 mm3, about 1 mm3 to about 5 mm3, about 1 mm3 to about 9 mm3, about 1 mm3 to about 10 mm3, about 1 mm3 to about 15 mm3, about 1 mm3 to about 20 mm3, about 4 mm3 to about 5 mm3, about 4 mm3 to about 9 mm3, about 4 mm3 to about 10 mm3, about 4 mm3 to about 15 mm3, about 4 mm3 to about 20 mm3, about 5 mm3 to about 9 mm3, about 5 mm3 to about 10 mm3, about 5 mm3 to about 15 mm3, about 5 mm3 to about 20 mm3, about 9 mm3 to about 10 mm3, about 9 mm3 to about 15 mm3, about 9 mm3 to about 20 mm3, about 10 mm3 to about 15 mm3, about 10 mm3 to about 20 mm3, or about 15 mm3 to about 20 mm3. In some embodiments, a composition as previously described comprises multiple types of spheroids, wherein the first type of spheroid, the second type of spheroid, and/or the third type of spheroid possess a volume from about 0.5 mm3, about 1 mm3, about 4 mm3, about 5 mm3, about 9 mm3, about 10 mm3, about 15 mm3, or about 20 mm3. In some embodiments, a composition as previously described comprises multiple types of spheroids, wherein the first type of spheroid, the second type of spheroid, and/or the third type of spheroid possess a volume from at least about 0.5 mm3, about 1 mm3, about 4 mm3, about 5 mm3, about 9 mm3, about 10 mm3, or about 15 mm3. In some embodiments, a composition as previously described comprises multiple types of spheroids, wherein the first type of spheroid, the second type of spheroid, and/or the third type of spheroid possess a volume from at most about 1 mm3, about 4 mm3, about 5 mm3, about 9 mm3, about 10 mm3, about 15 mm3, or about 20 mm3.

In some embodiments, a composition as previously described comprises a plurality of different types of spheroids, wherein the composition comprises a specific ratio of non-human animal cells selected from a group consisting of muscle cells and precursors, fat cells and precursors, connective tissue cells and precursors, cartilage cells and precursors, liver cells, heart cells, eye cells, kidney cells, skin cells, lung cells, or any combination thereof.

In some embodiments, a composition as previously described comprises a plurality of different types of spheroids, wherein the composition comprises a specific ratio of non-human animal cells selected from a group consisting of muscle cells and precursors, fat cells and precursors, connective tissue cells and precursors, cartilage cells and precursors, and combinations thereof. In some embodiments, a composition as previously described comprises a plurality of multiple types of spheroids, wherein the composition comprises a specific ratio of muscle cells to fat cells, muscle cells to connective tissue cells, or connective tissue cells to fat cells. In some embodiments, a composition as previously described comprises a plurality of multiple types of spheroids, wherein the composition comprises a specific ratio of muscle cells to fat cells to connective tissue cells. In some embodiments, a composition as previously described comprises a plurality of multiple types of spheroids, wherein the composition comprises a specific ratio of muscle cells to fat cells to connective tissue cells, further comprises cartilage.

In some embodiments, a spheroid used to make the compositions described herein comprises muscles cells and/or muscle cell precursors and is substantially free of cells of any other type. In some embodiments, a spheroid used to make the compositions described herein comprises a certain percentage of muscle cells and/or muscle cell precursors, wherein the percentage of muscle cells and/or muscle cell precursors is about 50% to about 95%, about 60% to about 95%, or about 75% to about 95%. In some embodiments, a spheroid comprises a percentage of muscle cells and/or muscle cell precursors, wherein the percentage of muscle cells and/or muscle cell precursors is about 50% to about 95%. In some embodiments, a spheroid comprises a percentage of muscle cells and/or muscle cell precursors, wherein the percentage of muscle cells is about 50% to about 99%. In certain embodiments, the percentage of muscle cells and/or muscle cell precursors is about 50% to about 60%, about 50% to about 70%, about 50% to about 75%, about 50% to about 80%, about 50% to about 85%, about 50% to about 90%, about 50% to about 95%, about 50% to about 97%, about 50% to about 99%, about 60% to about 70%, about 60% to about 75%, about 60% to about 80%, about 60% to about 85%, about 60% to about 90%, about 60% to about 95%, about 60% to about 97%, about 60% to about 99%, about 70% to about 75%, about 70% to about 80%, about 70% to about 85%, about 70% to about 90%, about 70% to about 95%, about 70% to about 97%, about 70% to about 99%, about 75% to about 80%, about 75% to about 85%, about 75% to about 90%, about 75% to about 95%, about 75% to about 97%, about 75% to about 99%, about 80% to about 85%, about 80% to about 90%, about 80% to about 95%, about 80% to about 97%, about 80% to about 99%, about 85% to about 90%, about 85% to about 95%, about 85% to about 97%, about 85% to about 99%, about 90% to about 95%, about 90% to about 97%, about 90% to about 99%, about 95% to about 97%, about 95% to about 99%, or about 97% to about 99%. In certain embodiments, the percentage of muscle cells and/or muscle cell precursors is about 50%, about 60%, about 70%, about 75%, about 80%, about 85%, about 90%, about 95%, about 97%, or about 99%. In certain embodiments, the percentage of muscle cells and/or muscle cell precursors is at least about 50%, about 60%, about 70%, about 75%, about 80%, about 85%, about 90%, about 95%, or about 97%. In certain embodiments, the percentage of muscle cells and/or muscle cell precursors is at most about 60%, about 70%, about 75%, about 80%, about 85%, about 90%, about 95%, about 97%, or about 99%.

In some embodiments, a spheroid used to make the compositions described herein comprises connective tissue cells and/or connective tissue cell precursors and is substantially free of cells of any other type. In some embodiments, a spheroid used to make the compositions described herein comprises a certain percentage of connective tissue cells, wherein the percentage of connective tissue cells of the spheroid is from about 1% to about 20%, about 1% to about 10%, or about 1% to about 5%. In some embodiments, a spheroid comprises a percentage of connective tissue cells and/or connective tissue cell precursors, wherein the percentage of connective tissue cells and/or connective tissue cell precursors of the spheroid is from about 1% to about 20%. In some embodiments, the percentage of connective tissue cells and/or connective tissue cell precursors is about 1% to about 2%, about 1% to about 5%, about 1% to about 7%, about 1% to about 10%, about 1% to about 12%, about 1% to about 15%, about 1% to about 17%, about 1% to about 20%, about 2% to about 5%, about 2% to about 7%, about 2% to about 10%, about 2% to about 12%, about 2% to about 15%, about 2% to about 17%, about 2% to about 20%, about 5% to about 7%, about 5% to about 10%, about 5% to about 12%, about 5% to about 15%, about 5% to about 17%, about 5% to about 20%, about 7% to about 10%, about 7% to about 12%, about 7% to about 15%, about 7% to about 17%, about 7% to about 20%, about 10% to about 12%, about 10% to about 15%, about 10% to about 17%, about 10% to about 20%, about 12% to about 15%, about 12% to about 17%, about 12% to about 20%, about 15% to about 17%, about 15% to about 20%, or about 17% to about 20%. In some embodiments, the percentage of connective tissue cells and/or connective tissue cell precursors is about 1%, about 2%, about 5%, about 7%, about 10%, about 12%, about 15%, about 17%, or about 20%. In some embodiments, the percentage of connective tissue cells and/or connective tissue cell precursors is at least about 1%, about 2%, about 5%, about 7%, about 10%, about 12%, about 15%, or about 17%. In some embodiments, the percentage of connective tissue cells and/or connective tissue cell precursors is at most about 2%, about 5%, about 7%, about 10%, about 12%, about 15%, about 17%, or about 20%.

In some embodiments, a spheroid used to make the compositions described herein comprises fat cells and is substantially free of cells of any other type. In some embodiments, a spheroid used to make the compositions described herein comprises a certain percentage of fat cells, wherein the percentage of fat cells is about 1% to about 50%, about 1% to about 30%, about 1% to about 20%, or about 1% to about 10%. In some embodiments, the percentage of fat cells is about 1% to about 50%. In some embodiments, the percentage of fat cells is about 1% to about 50%. In some embodiments, the percentage of fat cells is about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the percentage of fat cells is about 1%, about 5%, about 10%, about 15%, about 20%, about 25%, about 30%, about 35%, about 40%, about 45%, or about 50%. In some embodiments, the percentage of fat cells is at least about 1%, about 5%, about 10%, about 15%, about 20%, about 25%, about 30%, about 35%, about 40%, or about 45%. In some embodiments, the percentage of fat cells is at most about 5%, about 10%, about 15%, about 20%, about 25%, about 30%, about 35%, about 40%, about 45%, or about 50%.

Described herein are cultured meat product compositions comprising a plurality of types of spheroids. In some embodiments, the cultured meat product composition comprises an overall percentage of muscle cells and/or muscle cell precursors, wherein the percentage of muscle cells and/or muscle cell precursors is about 50% to about 95%, about 60% to about 95%, or about 75% to about 95%. In some embodiments, a composition comprises a percentage of muscle cells and/or muscle cell precursors, wherein the percentage of muscle cells and/or muscle cell precursors is about 50% to about 95%. In some embodiments, a composition comprises a percentage of muscle cells and/or muscle cell precursors, wherein the percentage of muscle cells and/or muscle cell precursors is about 50% to about 99%. In certain embodiments, the percentage of muscle cells and/or muscle cell precursors is about 50% to about 60%, about 50% to about 70%, about 50% to about 75%, about 50% to about 80%, about 50% to about 85%, about 50% to about 90%, about 50% to about 95%, about 50% to about 97%, about 50% to about 99%, about 60% to about 70%, about 60% to about 75%, about 60% to about 80%, about 60% to about 85%, about 60% to about 90%, about 60% to about 95%, about 60% to about 97%, about 60% to about 99%, about 70% to about 75%, about 70% to about 80%, about 70% to about 85%, about 70% to about 90%, about 70% to about 95%, about 70% to about 97%, about 70% to about 99%, about 75% to about 80%, about 75% to about 85%, about 75% to about 90%, about 75% to about 95%, about 75% to about 97%, about 75% to about 99%, about 80% to about 85%, about 80% to about 90%, about 80% to about 95%, about 80% to about 97%, about 80% to about 99%, about 85% to about 90%, about 85% to about 95%, about 85% to about 97%, about 85% to about 99%, about 90% to about 95%, about 90% to about 97%, about 90% to about 99%, about 95% to about 97%, about 95% to about 99%, or about 97% to about 99%. In certain embodiments, the percentage of muscle cells and/or muscle cell precursors is about 50%, about 60%, about 70%, about 75%, about 80%, about 85%, about 90%, about 95%, about 97%, or about 99%. In certain embodiments, the percentage of muscle cells and/or muscle cell precursors is at least about 50%, about 60%, about 70%, about 75%, about 80%, about 85%, about 90%, about 95%, or about 97%. In certain embodiments, the percentage of muscle cells and/or muscle cell precursors is at most about 60%, about 70%, about 75%, about 80%, about 85%, about 90%, about 95%, about 97%, or about 99%.

Described herein are cultured meat product compositions comprising a plurality of types of spheroids. In some embodiments, the cultured meat product composition comprises an overall percentage of connective tissue cells and/or connective tissue cell precursors, wherein the percentage of connective tissue cells and/or connective tissue cell precursors of the composition is from about 1% to about 20%, about 1% to about 10%, or about 1% to about 5%. In some embodiments, a composition comprises a percentage of connective tissue, wherein the percentage of connective tissue cells and/or connective tissue cell precursors of the composition is from about 1% to about 20%. In some embodiments, the percentage of connective tissue cells and/or connective tissue cell precursors is about 1% to about 2%, about 1% to about 5%, about 1% to about 7%, about 1% to about 10%, about 1% to about 12%, about 1% to about 15%, about 1% to about 17%, about 1% to about 20%, about 2% to about 5%, about 2% to about 7%, about 2% to about 10%, about 2% to about 12%, about 2% to about 15%, about 2% to about 17%, about 2% to about 20%, about 5% to about 7%, about 5% to about 10%, about 5% to about 12%, about 5% to about 15%, about 5% to about 17%, about 5% to about 20%, about 7% to about 10%, about 7% to about 12%, about 7% to about 15%, about 7% to about 17%, about 7% to about 20%, about 10% to about 12%, about 10% to about 15%, about 10% to about 17%, about 10% to about 20%, about 12% to about 15%, about 12% to about 17%, about 12% to about 20%, about 15% to about 17%, about 15% to about 20%, or about 17% to about 20%. In some embodiments, the percentage of connective tissue cells and/or connective tissue cell precursors is about 1%, about 2%, about 5%, about 7%, about 10%, about 12%, about 15%, about 17%, or about 20%. In some embodiments, the percentage of connective tissue cells and/or connective tissue cell precursors is at least about 1%, about 2%, about 5%, about 7%, about 10%, about 12%, about 15%, or about 17%. In some embodiments, the percentage of connective tissue cells and/or connective tissue cell precursors is at most about 2%, about 5%, about 7%, about 10%, about 12%, about 15%, about 17%, or about 20%.

Described herein are cultured meat product compositions comprising a plurality of types of spheroids. In some embodiments, the cultured meat product composition comprises an overall percentage of fat cells and/or fat cell precursors, wherein the percentage of fat cells and/or fat cell precursors is about 1% to about 50%, about 1% to about 30%, about 1% to about 20%, or about 1% to about 10%. In some embodiments, the percentage of fat cells and/or fat cell precursors is about 1% to about 50%. In some embodiments, the percentage of fat cells and/or fat cell precursors is about 1% to about 50%. In some embodiments, the percentage of fat cells and/or fat cell precursors is about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the percentage of fat cells and/or fat cell precursors is about 1%, about 5%, about 10%, about 15%, about 20%, about 25%, about 30%, about 35%, about 40%, about 45%, or about 50%. In some embodiments, the percentage of fat cells and/or fat cell precursors is at least about 1%, about 5%, about 10%, about 15%, about 20%, about 25%, about 30%, about 35%, about 40%, or about 45%. In some embodiments, the percentage of fat cells and/or fat cell precursors is at most about 5%, about 10%, about 15%, about 20%, about 25%, about 30%, about 35%, about 40%, about 45%, or about 50%.

In some embodiments, a composition as previously described comprises a plurality of multiple types of spheroids, wherein the composition comprises a specific ratio of non-human animal cells harvested from a tissue biopsy of a non-human animal. In some embodiments, a composition as previously described comprises a plurality of multiple types of spheroids, wherein the composition comprises non-human animal cells harvested from a tissue biopsy of a non-human animal. In some embodiments, a composition as previously described comprises a plurality of multiple types of spheroids, wherein the composition comprises a specific ratio of non-human animal cells harvested from a tissue biopsy of a non-human animal selected from the group consisting of: a cow, a pig, a chicken, a fish, a bird, a sheep, a bison, a wagyu, a boar, a reptile, an ostrich, a sheep, a goat, a camel, a duck, a goose, an elk, a deer, and a turkey. In some embodiments, the pig is selected from the group consisting of a Berkshire pig, a Kurobuta pig, and an Iberian pig.

In some embodiments, a composition as previously described comprises a plurality of multiple types of spheroids, wherein the composition comprises non-human animal cells selected from a group consisting of stem cells such as hematopoietic stem cells, mesenchymal stem cells, induced pluripotent stem cells, embryonic stem cells, adult stem cells, or any combination thereof. An extensive list of mammalian cell lines may be found in the American Type Culture Collection catalog (ATCC, Manassas, VA).

In some embodiments, a composition as previously described comprises a plurality of multiple types of spheroids, wherein the composition comprises a percentage of muscle cells, a percentage of fat cells, and a percentage of connective tissue cells that reproduce a percentage of muscle cells, a percentage of fat cells, and a percentage of connective tissue cells present in a beef sirloin, a beef ribeye, a beef short loin, a beef flank, a beef plate, a beef brisket, a beef rib, a beef round, a beef shank, a beef filet, a wagyu sirloin, a wagyu ribeye, a wagyu short loin, a wagyu flank, plate, a wagyu brisket, a wagyu rib, a wagyu round, a wagyu shank, a wagyu filet, a pork leg, a pork rib, a pork belly, a ham, a pork shoulder, a pork loin, a pork chop, a chicken wing, a chicken thigh, a chicken drumstick, a chicken breast, a duck wing, a duck thigh, a duck drumstick, a duck breast, a turkey wing, a turkey thigh, a turkey drumstick, a turkey breast, or any combination thereof. In some embodiments, a composition as previously described comprises a plurality of multiple types of spheroids, wherein the composition comprises a percentage of muscle cells, a percentage of fat cells, and a percentage of connective tissue cells that reproduce a percentage of muscle cells, a percentage of fat cells, and a percentage of connective tissue cells present in a wagyu sirloin, a wagyu ribeye, a wagyu short loin, a wagyu flank, plate, a wagyu brisket, a wagyu rib, a wagyu round, a wagyu shank, or a wagyu filet wherein the composition is equivalent to different quality grades of meat which can also comprise grading standards as related to marbling scores ranging from A1 to A5, B1 to B5, Cl to C5, or any combination thereof.

In some embodiments, a composition as previously described comprises a plurality of multiple types of spheroids, wherein the composition comprises a specific ratio of non-human animal cells or a specific ratio of spheroids that reproduce a pattern equivalent to marbled meat. In some embodiments, a composition as previously described comprises a plurality of multiple types of spheroids, wherein the composition comprises a specific ratio of non-human animal cells or a specific ratio of spheroids that produce marbled meat that is a beef steak. In some embodiments, after high-heat searing the composition comprises grill marks, sear marks, brown crust, marks equivalent to those of a cooked or partially cooked meat product, or any combination thereof. In some embodiments, after high-heat searing the composition comprises grill marks, sear marks, brown crust, or marks equivalent to those of a cooked or partially cooked beef steak.

In some embodiments, a composition as previously described comprises multiple types of spheroids, wherein the first type of spheroid, the second type of spheroid, and/or the third type of spheroid are cultured in vitro. In some embodiments, a composition as previously described comprises multiple types of spheroids, cultured in vitro.

In some embodiments, a composition as previously described comprises multiple types of spheroids further comprising one or more additives, including but not limited to, a flavoring, a flavor enhancer, a colorant, a color enhancer, a nutritional enhancer, or any combination thereof. In some embodiments, a composition comprises any combination of known flavoring or flavorant including meat flavors such as pork (e.g., 2-pyridine methanethiol), chicken, beef, veal, turkey, lamb, or any combination thereof; animal and non-animal fat or oil flavors (one or more fat or oil flavors such as fried fat, lard, tallow, chicken fat, bacon fat, turkey fat, pork fat, beef fat, sesame oil, olive oil, or any combination thereof); animal and non-animal dairy flavors (one or more dairy flavors such as cheese, cream, milk, sour cream, or any combination thereof), or any combination thereof. In some embodiments, a composition as previously described comprises a plurality of spheroids may be edible.

As defined herein, an “organoid” is a miniaturized and simplified version of an organ produced in vitro in 3D that shows realistic micro-anatomy. Organoids may be derived from one or a few cells from a tissue, stem cells, hematopoietic stem cells, mesenchymal stem cells, bone marrow cells, embryonic stem cells, induced pluripotent stem cells, precursor cells, or differentiated progenitor cells which can self-organize in three-dimensional culture owing to their self-renewal and differentiation capacities.

In some embodiments, a composition comprises a 3D formation or three-dimensional structure of spheroids comprises any of the spheroids described herein. In some embodiments, a composition comprises a 3D formation of a plurality of spheroids, wherein the spheroids are linked, at least partially linked, at least partially fused, loose aggregate, or any combination thereof. In some embodiments, a composition comprises a 3D formation of a plurality of spheroids comprises a plurality of cells, or a plurality of tissue such as muscle tissue, fat tissue, connective tissue, cartilage tissue, liver tissue, heart tissue, eye tissue, kidney tissue, endothelial tissue, lung tissue, or any combination thereof.

Spheroids can be grown via a few different methods. One common method of growing spheroids is to use low cell adhesion plates, typically a 96 well plate, to mass-produce spheroid cultures, where the aggregates form in the rounded bottom of the cell plates. Spheroids can also be cultured using a hanging drop method that involves forming cell aggregates in drops that hang from the surface of a cell plate. Other methods to grow spheroids may include the use of rotating wall vessel bioreactors, which spins and cultures the cells when they are constantly in free fall and forms aggregates in layers.

Cell spheroids are usually produced by using the hanging drop method mentioned previously. A fluid containing cells (e.g. eukaryotic cells, mammalian cells) is applied in individual droplets on an object slide in a hanging way. See, Ramsey Foty, “A simple hanging drop cell culture protocol for generation of 3D spheroids,” J. Vis. Exp. 2011, Vol. 51, Pages 2720, the entire contents of which are hereby incorporated by reference in their entirety. The cells tend to join each other, whereby cellular spheroids are formed in the droplets after a certain amount of time. Each droplet contains one cellular spheroid. However, such method to produce cellular spheroids has several drawbacks.

For example, surface tension limits the maximum size of a drop prepared by this method. Also, due to the small size of the suspended drops, evaporation is a large concern. As the water within the drop evaporates, the concentration of soluble components, such as proteins and salts in the medium, increases, subjecting the cells to a changing osmotic pressure, thus compromising their normal morphology and function. In order to avoid dehydration of the droplets and in order to supply additional cells and nutrients to the droplets to increase the size of the circular spheroids the droplets need to be carefully refilled from time to time. Most commonly, the droplets are refilled with a syringe having a fine needle. In order to avoid dripping of the droplets, refilling requires special care, since there is the risk that the droplets drop down from the object slide due to overfilling. In either way, an already formed cellular spheroid may be destroyed during cultivation.

Another disadvantage of this common production method is that only a few droplets can be applied to each object slide in order to avoid too much loss when the object slide is not handled carefully enough. Thus, a large number of individual object slides are necessary to produce a sufficient quantity of cellular spheroids.

3D spheroids have shown an adaptive response to recent advancements in micro-fluidic technologies, which has allowed better control over spheroid sizes and subsequent drug screening studies. As defined herein, “micro-fluidic technology” is the adaptation, miniaturization, integration, and automation of analytical laboratory procedures into a single device or “chip”. Numerous micro-fluidic chips, systems, and devices for cultivation of cellular spheroids are known in the art. See, K. Kwapiszewska, et al., “A microfluidic-based platform for tumor spheroid culture, monitoring and drug screening,” Lab on a Chip, 2014, Vol. 14, Issue 12, Pages 2096-2104; Agnieszka Zuchowska, et al., “Studies of anticancer drug cytotoxicity based on long-term HepG2 spheroid culture in a microfluidic system,” Electrophoresis, 2017, Vol. 38, Issue 8, Pages 1206-1216; Bishnubrata Patra, et al., “A microfluidic device for uniform-sized cell spheroids formation, culture, harvesting and flow cytometry analysis,” Biomicrofluidics, 2013, Vol. 7, Pages 054114-1 to 054114-11; Khashayar Moshksayan, et al., “Spheroids-on-a-chip: Recent advances and design considerations in microfluidic platforms for spheroid formation and culture,” Sensors and Actuators B, 2018, Vol. 263, Pages 151-176, U.S. Published Patent Application No. 2016/097028 A1 published on Apr. 7, 2016; WO 2017/188890 A1 published on Nov. 2, 2017; WO 2018/067802 A1 published on Apr. 12, 2018; U.S. Published Patent Application No. 2009/298116 A1 published on Dec. 3, 2009; and WO 2020/058490 A1 published on Mar. 26, 2020, the entire contents of which are hereby incorporated by reference in their entirety.

The micro-fluidic device of the present invention may be a micro-fluidic chip or a microfluidics chip, used interchangeably, in examples, that may be used for the production of cellular spheroids. The device may include at least one chamber comprising a fluid inlet for introducing fluid into the chamber and a fluid outlet for removing the fluid from the chamber. The at least one chamber comprises a base formed by a substrate comprising at least two recesses to collect a fluid comprising biological cells when the substrate is contacted with the fluid. The size of the at least two recesses decreases from the fluid inlet to the fluid outlet of the at least one chamber. One major advantage of the micro-fluidic device of the present invention is the possibility to produce cellular spheroids having a defined size/diameter in a more reproducible manner compared to other techniques like hanging drop cell cultures. See, Foty.

Another advantage using the micro-fluidic device of the present invention to produce cellular spheroids is the possibility to supply the cells with fresh culture medium during spheroid formation by simply providing a flow of culture medium during the production. The provision of at least two recesses in the substrate of the device of the present invention allows the production of a random number of circular spheroids within one microfluidic device. Furthermore, by providing a plurality of recesses on the substrate, cellular spheroids can be produced cost-efficiently.

Preferably, the microfluidic device according to the invention has between 15 and 30 recesses. The at least two recesses have a different size and shape. This has the advantage, that different sized cellular spheroids can be produced within one single microfluidic device. The size of the at least two recesses decreases from the fluid inlet to the fluid outlet of the at least one chamber. Therefore, the smallest recess/recesses is/are located closest to the fluid outlet.

By rinsing the at least one chamber with the discharging fluid at different velocities, the cellular spheroids can be eluted according to their size. Since the smallest recess/recesses is/are arranged closest to the outlet and the rest of the recesses are arranged in an ascending manner towards the inlet, the advantage is obtained that by eluting the cellular spheroids according to their size starting out with the smallest recess/recesses eluted cellular spheroids cannot get stuck in other recesses still containing cellular spheroids.

The substrate of the micro-fluidic device comprises, consists of or is coated at least partially with a biocompatible material. The biocompatible material is a silicone or a plastic material or glass. In particular, the biocompatible material may be: polystyrene, cycloolefin-copolymer, polymethylmethacrylate, cyclo-olefin polymer, polydimethylsiloxane, polycarbonate (PC), polypropylene (PP), polyvinylchloride (PVC), perflouropolyether (PFPE), polyurethane, poly(ethyleneterephthalate) (PET), polyester and thiol-enes. Using a biocompatible material for the substrate or coating the substrate with a biocompatible material has the advantage, that the cells of the cellular spheroid are not affected and hence probably intoxicated by the substrate.

In some embodiments, the cells are cultivated in plates comprising, consisting of or coated at least partially with a heterologous extracellular matrix. In some embodiments, the cells are cultivated in plates comprising, consisting of or coated at least partially with a biocompatible material like fibers or hydrogels. In some embodiments, plates comprising fiber or nanofibers are exemplary confinement materials possessing one or more advantageous properties including biocompatible, optically transparent adjustable fibers, compatible with 3D and 2D cell culture, mimicry of 3D topography, or any combination thereof. In some embodiments, the optically transparent fibers allow for live-cell imaging and real time quantification of cell mobility of 3D cell culture. In some embodiments, the optically transparent fibers allow for live-cell imaging and real time quantification of cell mobility of 3D or 2D cell culture.

In some embodiments, the composition comprises a mixture of 3D cells and 2D cells. In some embodiments, the composition comprises a mixture of spheroids or organoids and 2D cells. In some embodiments, the composition comprises 3D printed of spheroids, organoids, a mixture of spheroids/organoids, or any combination thereof, further mixed with 2D cells. In some embodiments, the method comprises 3D printing of spheroids, organoids, a mixture of spheroids/organoids, or any combination thereof, further mixed with 2D cells.

In some embodiments, hydrogels are exemplary confinement materials possessing one or more advantageous properties including: non-adherent, biocompatible, extrudable, bioprintable, non-cellular, of suitable strength, and not soluble in aqueous conditions. In some embodiments, suitable hydrogels are natural polymers. In one embodiment, the confinement material is comprised of NovoGel™. In further embodiments, suitable hydrogels include those derived from surfactant polyols such as Pluronic F-127, collagen, hyaluronate, fibrin, alginate, agarose, chitosan, and derivatives or combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel™, agarose, alginate, gelatin, Matrigel™ (e.g., solubilized basement membrane matrix secreted by Engelbreth-Holm-Swarm (EHS) mouse sarcoma cells, Corning Life sciences), hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, and combinations thereof.

In some embodiments, the cells are cultivated in plates, flasks or dishes compatible with cell culture comprising, consisting of or coated at least partially with a biocompatible material like a heterologous extracellular matrix. In some embodiments, the cells are cultivated in plates comprising, consisting of or coated at least partially with a biocompatible material like a heterologous extracellular matrix comprising 5-15% gelatinous protein mixture (for example secreted by Engelbreth-Holm-Swarm mouse sarcoma cells, also referred to as Matrigel). In some embodiments, the cells are cultivated in plates comprising, consisting of or coated at least partially with a biocompatible material like a heterologous extracellular matrix comprising 5-15% Matrigel™ or laminin. In some embodiments, a composition as previously described comprising multiple types of cells, wherein the cells are cultivated in plates comprising, consisting of, or coated at least partially with heterologous extracellular matrix comprising a volume of Matrigel™ ranging from about 1% to about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from about 5% to about 15%. In some embodiments, a composition as previously described comprising multiple types of cells, wherein the cells are cultivated in plates comprising, consisting of, or coated at least partially with heterologous extracellular matrix comprising a volume of Matrigel™ ranging from about 6% to about 14%. In some embodiments, a composition as previously described comprising multiple types of cells, is cultivated in plates comprising, consisting of, or coated at least partially with heterologous extracellular matrix comprising a volume of Matrigel™ ranging from about 1% to about 5%, about 1% to about 7%, about 1% to about 10%, about 1% to about 12%, about 1% to about 15%, about 1% to about 20%, about 1% to about 25%, about 5% to about 7%, about 5% to about 10%, about 5% to about 12%, about 5% to about 15%, about 5% to about 20%, about 5% to about 25%, about 7% to about 10%, about 7% to about 12%, about 7% to about 15%, about 7% to about 20%, about 7% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from about 1%, about 5%, about 7%, about 10%, about 12%, about 15%, about 20%, or about 25%. In some embodiments, a composition as previously described comprising multiple types of cells, wherein the cells are cultivated in plates comprising, consisting of, or coated at least partially with heterologous extracellular matrix comprising a volume of Matrigel™ ranging from at least about 1%, about 5%, about 7%, about 10%, about 12%, about 15%, or about 20%. In some embodiments, a composition as previously described comprising multiple types of cells, wherein the cells are cultivated in plates comprising, consisting of, or coated at least partially with heterologous extracellular matrix comprising a volume of Matrigel™ ranging from at most about 5%, about 7%, about 10%, about 12%, about 15%, about 20%, or about 25%. The biocompatible material of the plates dishes or flasks may comprise suitable hydrogels that include those derived from surfactant polyols such as Pluronic F-127, collagen, hyaluronate, fibrin, alginate, agarose, chitosan, and derivatives or combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel™, agarose, alginate, gelatin, Matrigel™ (e.g., solubilized basement membrane matrix secreted by Engelbreth-Holm-Swarm (EHS) mouse sarcoma cells, Corning Life sciences), hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, and combinations thereof.

Plates, dishes or flasks for culturing the cells and/or spheroids described herein may comprise one or more recesses. The plates, dishes, and/or flasks may comprise at least two recesses. Preferably, the at least two recesses have the shape of a hemisphere, a spherical cap, a semi ellipsoid, a cone, a truncated cone, a terraced cone, a pyramid, a truncated pyramid, a terraced pyramid, a torus, or an elliptic paraboloid, among other shapes. Especially with two recesses in the shape of a hemisphere, round cellular spheroids can be produced with the microfluidic according to the invention. With the at least two recesses having the shape of a spherical cap, the spherical cap has a polar angle a of 30° to 90°, preferably 40° to 90°, more preferably 50° to 90°, more preferably 60° to 90°, more preferably 70° to 90°, more preferably to 90°, and more preferably 85° to 90°. The plates, dishes or flasks may comprise at least 3, 4, 5, 6, 7, 8, 9, 10, 11, 12, 24, 48, 96, or 384 recesses.

The at least two recesses have a depth of 50 pm to 1 mm, preferably 50 pm to 800 pm, and more preferably 50 pm to 500 pm. The at least two recesses have a width or diameter of 100 pm to 2 mm, preferably 100 pm to 1.5 mm, and more preferably 100 pm to 1 mm. Advantageously, the ratio of depth to width of the at least two recesses is between 1 to 1 and 1 to 3, and preferably 1 to 1 to 1 to 2.

Practically, the at least two recesses are spaced apart from each other at a distance from pm to 6 mm, preferably 20 pm to 6 mm, more preferably 30 pm to 6 mm, more preferably 50 pm to 6 mm, more preferably 70 pm to 6 mm, more preferably 100 pm to 6 mm, and more preferably 100 pm to 5 mm more preferably 100 pm to 4 mm.

In some embodiments, the at least two recesses possess a dimeter from about 50 micrometers to about 1,000 micrometers. In some embodiments, the at least two recesses possess a dimeter from at least about 50 micrometers. In some embodiments, the at least two recesses possess a dimeter from at most about 1,000 micrometers. In some embodiments, the at least two recesses possess a dimeter from about 50 micrometers to about 100 micrometers, about micrometers to about 200 micrometers, about 50 micrometers to about 300 micrometers, about 50 micrometers to about 400 micrometers, about 50 micrometers to about 500 micrometers, about 50 micrometers to about 600 micrometers, about 50 micrometers to about 700 micrometers, about 50 micrometers to about 800 micrometers, about 50 micrometers to about 900 micrometers, about 50 micrometers to about 1,000 micrometers, about 100 micrometers to about 200 micrometers, about 100 micrometers to about 300 micrometers, about 100 micrometers to about 400 micrometers, about 100 micrometers to about 500 micrometers, about 100 micrometers to about 600 micrometers, about 100 micrometers to about 700 micrometers, about 100 micrometers to about 800 micrometers, about 100 micrometers to about 900 micrometers, about 100 micrometers to about 1,000 micrometers, about 200 micrometers to about 300 micrometers, about 200 micrometers to about 400 micrometers, about 200 micrometers to about 500 micrometers, about 200 micrometers to about 600 micrometers, about 200 micrometers to about 700 micrometers, about 200 micrometers to about 800 micrometers, about 200 micrometers to about 900 micrometers, about 200 micrometers to about 1,000 micrometers, about 300 micrometers to about 400 micrometers, about 300 micrometers to about 500 micrometers, about 300 micrometers to about 600 micrometers, about 300 micrometers to about 700 micrometers, about 300 micrometers to about 800 micrometers, about 300 micrometers to about 900 micrometers, about 300 micrometers to about 1,000 micrometers, about 400 micrometers to about 500 micrometers, about 400 micrometers to about 600 micrometers, about 400 micrometers to about 700 micrometers, about 400 micrometers to about 800 micrometers, about 400 micrometers to about 900 micrometers, about 400 micrometers to about 1,000 micrometers, about 500 micrometers to about 600 micrometers, about 500 micrometers to about 700 micrometers, about 500 micrometers to about 800 micrometers, about 500 micrometers to about 900 micrometers, about 500 micrometers to about 1,000 micrometers, about 600 micrometers to about 700 micrometers, about 600 micrometers to about 800 micrometers, about 600 micrometers to about 900 micrometers, about 600 micrometers to about 1,000 micrometers, about 700 micrometers to about 800 micrometers, about 700 micrometers to about 900 micrometers, about 700 micrometers to about 1,000 micrometers, about 800 micrometers to about 900 micrometers, about 800 micrometers to about 1,000 micrometers, or about 900 micrometers to about 1,000 micrometers. Preferably, the at least one cellular spheroid formed in at least two recesses is eluted by rinsing the at least one chamber with a discharging fluid at a velocity sufficient to elute the cellular spheroid. Preferably, with a substrate comprising at least two recesses of different size the cellular spheroids are eluted by successively rinsing the at least one chamber with a discharging fluid at different velocities. This has the advantage that the cellular spheroids are already sorted according to their size after elution. A subsequent separation is therefore not necessary. The at least two recess may have the same diameter or a different diameter.

Another aspect the present invention relates to the production of cellular spheroids comprising the steps of applying a fluid comprising biological cells into at least one chamber of a device according to the invention and thus providing said fluid to the at least two recesses and incubating the device comprising said fluid for at least 2 hours until at least one cellular spheroid is formed in the at least two recesses. These and other specifics regarding the micro-fluidic device find support in WO 2020/058490 A1 published on Mar. 26, 2020, the entire contents of which are hereby incorporated by reference in their entirety.

Another aspect the present invention relates to the production of cellular spheroids comprising different metabolic activity following treatment with recombinant growth factors. In some embodiments, spheroids are treated with non-human recombinant growth factors or growth factors of the species from which the cells are sourced. In some embodiments, spheroids are treated with recombinant growth factors selected from the group consisting of vascular endothelial growth factor (VEGF (A-F)), fibroblast growth factors (acidic and basic FGF 1-10), granulocyte-macrophage colony-stimulating factor (GM-CSF), insulin, insulin growth factor or insulin-like growth factor (IGF), insulin growth factor binding protein (IGFBP), placenta growth factor (PIGF), angiopoietin (Ang1 and Ang2), platelet-derived growth factor (PDGF), hepatocyte growth factor (HGF), transforming growth factor (TGF-α, TGF-β, isoforms 1-3), platelet-endothelial cell adhesion molecule-1 (PECAM-1), vascular endothelial cadherin (VE-cadherin), nitric oxide (NO), chemokine (C-X-C motif) ligand 10 (CXCL10) or IP-10, interleukin-8 (IL-8), hypoxia inducible factor (HIF), monocyte chemotactic protein-1 (MCP-1), vascular cell adhesion molecule (VCAM), ephrin ligands (including Ephrin-B2 and -B4). Transcription factors include, but are not limited to, HIF-1α, HIF-1β and HIF-2α, Ets-1, Hex, Vezf1, Hox, GATA, LKLF, COUP-TFII, Hox, MEF2, Braf, Prx-1, Prx-2, CRP2/SmLIM and GATA family members, basic helix-loop-helix factors, or any combination thereof. In some embodiments, spheroids are treated with recombinant growth factors selected from the group consisting of fibroblast growth factor (FGF), hepatocyte growth factor (HGF), and insulin-like growth factor (IGF). In some embodiments, spheroids are treated with one of these recombinant growth factors selected from the group consisting of fibroblast growth factor (FGF), hepatocyte growth factor (HGF), and insulin-like growth factor (IGF). In some embodiments, spheroids are treated with any of these recombinant growth factors selected from the group consisting of fibroblast growth factor (FGF), hepatocyte growth factor (HGF), and insulin-like growth factor (IGF). In some embodiments, spheroids are treated with fibroblast growth factor (FGF). In some embodiments, spheroids are treated with hepatocyte growth factor (HGF). In some embodiments, spheroids are treated with insulin-like growth factor (IGF).

The spheroids described herein can have superior metabolic activity as tested by

In some embodiments, spheroids manufactured according to the methods described herein will utilize one or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize two or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize three or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize four or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize five or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize six or more growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). In some embodiments, spheroids manufactured according to the methods described herein will utilize all seven growth factors selected form the list consisting of hepatocyte growth factor (HGF), fibroblast growth factor (FGF), insulin-like growth factor (IGF), epidermal growth factor (EGF), transforming growth factor beta (TGFbeta), platelet-derived growth factor (PDGF), and vascular endothelial growth factor (VEGF). The one or more growth factors can be included in the concentrations described below and can be added before or during spheroid formation. In certain embodiments, the growth factors are supplied after individual cells of a certain cell type have been harvested (e.g., muscle, fat, or connective tissue cells) and placed in a culture vessel, on a plate, or on a microfluidics chip to facilitate formation of spheroids or organoids.

The growth factor cultured with the spheroids or cells or cell precursors described herein can be derived from the same animal or species as the spheroids, cells, or cell precursors, or from a different animal or species as the spheroids, cells, or cell precursors. The growth factors can be recombinantly produced.

In some embodiments, a composition as described comprises one type or a plurality of types of spheroids, wherein the spheroids are treated with a concentration of fibroblast growth factor (FGF). In certain embodiments, the FGF is at a concentration ranging from about 1 ng/mL to about 20 ng/mL. In some embodiments, a composition as previously described comprising multiple types of spheroids, wherein the spheroids are treated with a concentration of fibroblast growth factor (FGF) of about 8 ng/mL. In some embodiments, a composition as previously described comprising multiple types of spheroids, wherein the spheroids are treated with a concentration of fibroblast growth factor (FGF) ranging from about 1 ng/mL to about 4 ng/mL, about 1 ng/mL to about 5 ng/mL, about 1 ng/mL to about 8 ng/mL, about 1 ng/mL to about 10 ng/mL, about 1 ng/mL to about 15 ng/mL, about 1 ng/mL to about 18 ng/mL, about 1 ng/mL to about 20 ng/mL, about 4 ng/mL to about 5 ng/mL, about 4 ng/mL to about 8 ng/mL, about 4 ng/mL to about 10 ng/mL, about 4 ng/mL to about 15 ng/mL, about 4 ng/mL to about 18 ng/mL, about 4 ng/mL to about 20 ng/mL, about 5 ng/mL to about 8 ng/mL, about 5 ng/mL to about 10 ng/mL, about 5 ng/mL to about 15 ng/mL, about 5 ng/mL to about 18 ng/mL, about 5 ng/mL to about 20 ng/mL, about 8 ng/mL to about 10 ng/mL, about 8 ng/mL to about 15 ng/mL, about 8 ng/mL to about 18 ng/mL, about 8 ng/mL to about 20 ng/mL, about 10 ng/mL to about 15 ng/mL, about 10 ng/mL to about 18 ng/mL, about 10 ng/mL to about 20 ng/mL, about 15 ng/mL to about 18 ng/mL, about 15 ng/mL to about 20 ng/mL, or about 18 ng/mL to about 20 ng/mL. In some embodiments, a composition as previously described comprising multiple types of spheroids, wherein the spheroids are treated with a concentration of fibroblast growth factor (FGF) ranging from about 1 ng/mL, about 4 ng/mL, about 5 ng/mL, about 8 ng/mL, about 10 ng/mL, about 15 ng/mL, about 18 ng/mL, or about 20 ng/mL. In some embodiments, a composition as previously described comprising multiple types of spheroids, wherein the spheroids are treated with a concentration of fibroblast growth factor (FGF) ranging from at least about 1 ng/mL, about 4 ng/mL, about 5 ng/mL, about 8 ng/mL, about 10 ng/mL, about 15 ng/mL, or about 18 ng/mL. In some embodiments, a composition as previously described comprising multiple types of spheroids, wherein the spheroids are treated with a concentration of fibroblast growth factor (FGF) ranging from at most about 4 ng/mL, about 5 ng/mL, about 8 ng/mL, about 10 ng/mL, about 15 ng/mL, about 18 ng/mL, or about 20 ng/mL. Spheroids may be treated for about 1, 2, 3, 4, 5, 6, 7, 8, 9, 10, 11, 12, 13, 14, or more days with FGF.

In some embodiments, a composition described comprises one type or a plurality of types of spheroids, wherein the spheroids are treated with a concentration of hepatocyte growth factor (HGF). In certain embodiments, the HGF is at a concentration ranging from about 5 ng/mL to about 100 ng/mL. In some embodiments, a composition as previously described comprising multiple types of spheroids, wherein the spheroids are treated with a concentration of hepatocyte growth factor (HGF) of about 40 ng/mL. In some embodiments, a composition as previously described comprising multiple types of spheroids, wherein the spheroids are treated with a concentration of hepatocyte growth factor (HGF) ranging from about 5 ng/mL to about 10 ng/mL, about 5 ng/mL to about 20 ng/mL, about 5 ng/mL to about 40 ng/mL, about 5 ng/mL to about 50 ng/mL, about 5 ng/mL to about 80 ng/mL, about 5 ng/mL to about 100 ng/mL, about ng/mL to about 20 ng/mL, about 10 ng/mL to about 40 ng/mL, about 10 ng/mL to about 50 ng/mL, about 10 ng/mL to about 80 ng/mL, about 10 ng/mL to about 100 ng/mL, about 20 ng/mL to about 40 ng/mL, about 20 ng/mL to about 50 ng/mL, about 20 ng/mL to about 80 ng/mL, about 20 ng/mL to about 100 ng/mL, about 40 ng/mL to about 50 ng/mL, about 40 ng/mL to about 80 ng/mL, about 40 ng/mL to about 100 ng/mL, about 50 ng/mL to about 80 ng/mL, about 50 ng/mL to about 100 ng/mL, or about 80 ng/mL to about 100 ng/mL. In some embodiments, a composition as previously described comprising multiple types of spheroids, wherein the spheroids are treated with a concentration of hepatocyte growth factor (HGF) ranging from about 5 ng/mL, about 10 ng/mL, about 20 ng/mL, about 40 ng/mL, about 50 ng/mL, about 80 ng/mL, or about 100 ng/mL. In some embodiments, a composition as previously described comprising multiple types of spheroids, wherein the spheroids are treated with a concentration of hepatocyte growth factor (HGF) ranging from at least about 5 ng/mL, about 10 ng/mL, about 20 ng/mL, about 40 ng/mL, about 50 ng/mL, or about 80 ng/mL. In some embodiments, a composition as previously described comprising multiple types of spheroids, wherein the spheroids are treated with a concentration of hepatocyte growth factor (HGF) ranging from at most about 10 ng/mL, about 20 ng/mL, about 40 ng/mL, about 50 ng/mL, about 80 ng/mL, or about 100 ng/mL. Spheroids may be treated for about 1, 2, 3, 4, 5, 6, 7, 8, 9, 10, 11, 12, 13, 14, or more days with HGF.

In some embodiments, a composition described comprises one type or a plurality of types of spheroids, wherein the spheroids are treated with a concentration of insulin-like growth factor (IGF). In certain embodiments, the IGF is at a concentration ranging from about 1 ng/mL to about 50 ng/mL. In some embodiments, a composition as previously described comprising multiple types of spheroids, wherein the spheroids are treated with a concentration of insulin-like growth factor (IGF) of about 20 ng/mL. In some embodiments, a composition as previously described comprising multiple types of spheroids, wherein the spheroids are treated with a concentration of insulin-like growth factor (IGF) ranging from about 1 ng/mL to about 5 ng/mL, about 1 ng/mL to about 10 ng/mL, about 1 ng/mL to about 20 ng/mL, about 1 ng/mL to about 40 ng/mL, about 1 ng/mL to about 50 ng/mL, about 5 ng/mL to about 10 ng/mL, about 5 ng/mL to about 20 ng/mL, about 5 ng/mL to about 40 ng/mL, about 5 ng/mL to about 50 ng/mL, about 10 ng/mL to about 20 ng/mL, about 10 ng/mL to about 40 ng/mL, about 10 ng/mL to about 50 ng/mL, about 20 ng/mL to about 40 ng/mL, about 20 ng/mL to about 50 ng/mL, or about 40 ng/mL to about 50 ng/mL. In some embodiments, a composition as previously described comprising multiple types of spheroids, wherein the spheroids are treated with a concentration of insulin-like growth factor (IGF) ranging from about 1 ng/mL, about 5 ng/mL, about 10 ng/mL, about 20 ng/mL, about 40 ng/mL, or about 50 ng/mL. In some embodiments, a composition as previously described comprising multiple types of spheroids, wherein the spheroids are treated with a concentration of insulin-like growth factor (IGF) ranging from at least about 1 ng/mL, about 5 ng/mL, about 10 ng/mL, about 20 ng/mL, or about 40 ng/mL. In some embodiments, a composition as previously described comprising multiple types of spheroids, wherein the spheroids are treated with a concentration of insulin-like growth factor (IGF) ranging from at most about 5 ng/mL, about 10 ng/mL, about 20 ng/mL, about 40 ng/mL, or about 50 ng/mL. Spheroids may be treated for about 1, 2, 3, 4, 5, 6, 7, 8, 9, 10, 11, 12, 13, 14, or more days with IGF.

In some embodiments, a composition described comprises one type or a plurality of types of spheroids, wherein the spheroids are treated with a concentration of epiderma growth factor (EGF). In certain embodiments, the EGF is at a concentration ranging from about 0.1 ng/mL to about 50 ng/mL. In some embodiments, a composition as previously described comprising multiple types of spheroids, wherein the spheroids are treated with a concentration of insulin-like growth factor (IGF) of about 20 ng/mL. In some embodiments, spheroids are treated with a concentration of EGF of about 0.1 ng/mL to about 50 ng/mL. In some embodiments, spheroids are treated with a concentration of EGF of about 0.1 ng/mL to about 0.5 ng/mL, about 0.1 ng/mL to about 1 ng/mL, about 0.1 ng/mL to about 2 ng/mL, about 0.1 ng/mL to about 5 ng/mL, about 0.1 ng/mL to about 10 ng/mL, about 0.1 ng/mL to about 15 ng/mL, about 0.1 ng/mL to about 20 ng/mL, about 0.1 ng/mL to about 25 ng/mL, about 0.1 ng/mL to about 50 ng/mL, about 0.5 ng/mL to about 1 ng/mL, about 0.5 ng/mL to about 2 ng/mL, about ng/mL to about 5 ng/mL, about 0.5 ng/mL to about 10 ng/mL, about 0.5 ng/mL to about 15 ng/mL, about 0.5 ng/mL to about 20 ng/mL, about 0.5 ng/mL to about 25 ng/mL, about 0.5 ng/mL to about 50 ng/mL, about 1 ng/mL to about 2 ng/mL, about 1 ng/mL to about 5 ng/mL, about 1 ng/mL to about 10 ng/mL, about 1 ng/mL to about 15 ng/mL, about 1 ng/mL to about 20 ng/mL, about 1 ng/mL to about 25 ng/mL, about 1 ng/mL to about 50 ng/mL, about 2 ng/mL to about 5 ng/mL, about 2 ng/mL to about 10 ng/mL, about 2 ng/mL to about 15 ng/mL, about 2 ng/mL to about 20 ng/mL, about 2 ng/mL to about 25 ng/mL, about 2 ng/mL to about 50 ng/mL, about 5 ng/mL to about 10 ng/mL, about 5 ng/mL to about 15 ng/mL, about 5 ng/mL to about 20 ng/mL, about 5 ng/mL to about 25 ng/mL, about 5 ng/mL to about 50 ng/mL, about 10 ng/mL to about 15 ng/mL, about 10 ng/mL to about 20 ng/mL, about 10 ng/mL to about 25 ng/mL, about ng/mL to about 50 ng/mL, about 15 ng/mL to about 20 ng/mL, about 15 ng/mL to about 25 ng/mL, about 15 ng/mL to about 50 ng/mL, about 20 ng/mL to about 25 ng/mL, about 20 ng/mL to about 50 ng/mL, or about 25 ng/mL to about 50 ng/mL. In some embodiments, spheroids are treated with a concentration of EGF of about 0.1 ng/mL, about 0.5 ng/mL, about 1 ng/mL, about 2 ng/mL, about 5 ng/mL, about 10 ng/mL, about 15 ng/mL, about 20 ng/mL, about 25 ng/mL, or about 50 ng/mL. In some embodiments, spheroids are treated with a concentration of EGF of at least about 0.1 ng/mL, about 0.5 ng/mL, about 1 ng/mL, about 2 ng/mL, about 5 ng/mL, about ng/mL, about 15 ng/mL, about 20 ng/mL, or about 25 ng/mL. In some embodiments, spheroids are treated with a concentration of EGF of at most about 0.5 ng/mL, about 1 ng/mL, about 2 ng/mL, about 5 ng/mL, about 10 ng/mL, about 15 ng/mL, about 20 ng/mL, about 25 ng/mL, or about 50 ng/mL. Spheroids may be treated for about 1, 2, 3, 4, 5, 6, 7, 8, 9, 10, 11, 12, 13, 14, or more days with EGF.

In some embodiments, a composition described comprises one type or a plurality of types of spheroids, wherein the spheroids are treated with a concentration of transforming growth factor beta (TGFbeta). In some embodiments, spheroids are treated with a concentration of TGFbeta of about 0.5 ng/mL to about 20 ng/mL. In some embodiments, spheroids are treated with a concentration of TGFbeta of about 0.5 ng/mL to about 1 ng/mL, about 0.5 ng/mL to about 2 ng/mL, about 0.5 ng/mL to about 3 ng/mL, about 0.5 ng/mL to about 4 ng/mL, about 0.5 ng/mL to about 5 ng/mL, about 0.5 ng/mL to about 6 ng/mL, about 0.5 ng/mL to about 7 ng/mL, about 0.5 ng/mL to about 8 ng/mL, about 0.5 ng/mL to about 9 ng/mL, about 0.5 ng/mL to about 10 ng/mL, about 0.5 ng/mL to about 20 ng/mL, about 1 ng/mL to about 2 ng/mL, about 1 ng/mL to about 3 ng/mL, about 1 ng/mL to about 4 ng/mL, about 1 ng/mL to about 5 ng/mL, about 1 ng/mL to about 6 ng/mL, about 1 ng/mL to about 7 ng/mL, about 1 ng/mL to about 8 ng/mL, about 1 ng/mL to about 9 ng/mL, about 1 ng/mL to about 10 ng/mL, about 1 ng/mL to about 20 ng/mL, about 2 ng/mL to about 3 ng/mL, about 2 ng/mL to about 4 ng/mL, about 2 ng/mL to about 5 ng/mL, about 2 ng/mL to about 6 ng/mL, about 2 ng/mL to about 7 ng/mL, about 2 ng/mL to about 8 ng/mL, about 2 ng/mL to about 9 ng/mL, about 2 ng/mL to about 10 ng/mL, about 2 ng/mL to about 20 ng/mL, about 3 ng/mL to about 4 ng/mL, about 3 ng/mL to about 5 ng/mL, about 3 ng/mL to about 6 ng/mL, about 3 ng/mL to about 7 ng/mL, about 3 ng/mL to about 8 ng/mL, about 3 ng/mL to about 9 ng/mL, about 3 ng/mL to about 10 ng/mL, about 3 ng/mL to about 20 ng/mL, about 4 ng/mL to about 5 ng/mL, about 4 ng/mL to about 6 ng/mL, about 4 ng/mL to about 7 ng/mL, about 4 ng/mL to about 8 ng/mL, about 4 ng/mL to about 9 ng/mL, about 4 ng/mL to about 10 ng/mL, about 4 ng/mL to about 20 ng/mL, about 5 ng/mL to about 6 ng/mL, about 5 ng/mL to about 7 ng/mL, about 5 ng/mL to about 8 ng/mL, about 5 ng/mL to about 9 ng/mL, about 5 ng/mL to about 10 ng/mL, about 5 ng/mL to about 20 ng/mL, about 6 ng/mL to about 7 ng/mL, about 6 ng/mL to about 8 ng/mL, about 6 ng/mL to about 9 ng/mL, about 6 ng/mL to about 10 ng/mL, about 6 ng/mL to about 20 ng/mL, about 7 ng/mL to about 8 ng/mL, about 7 ng/mL to about 9 ng/mL, about 7 ng/mL to about 10 ng/mL, about 7 ng/mL to about 20 ng/mL, about 8 ng/mL to about 9 ng/mL, about 8 ng/mL to about 10 ng/mL, about 8 ng/mL to about 20 ng/mL, about 9 ng/mL to about 10 ng/mL, about 9 ng/mL to about 20 ng/mL, or about 10 ng/mL to about 20 ng/mL. In some embodiments, spheroids are treated with a concentration of TGFbeta of about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, or about 20 ng/mL. In some embodiments, spheroids are treated with a concentration of TGFbeta of at least about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, or about 10 ng/mL. In some embodiments, spheroids are treated with a concentration of TGFbeta of at most about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, or about 20 ng/mL. Spheroids may be treated for about 1, 2, 3, 4, 5, 6, 7, 8, 9, 10, 11, 12, 13, 14, or more days with TGFbeta.

In some embodiments, a composition described comprises one type or a plurality of types of spheroids, wherein the spheroids are treated with a concentration of platelet derived growth factor beta (PDGF). In some embodiments, spheroids are treated with a concentration of PDGF of about 1 ng/mL to about 100 ng/mL. In some embodiments, spheroids are treated with a concentration of PDGF of about 1 ng/mL to about 2 ng/mL, about 1 ng/mL to about 5 ng/mL, about 1 ng/mL to about 10 ng/mL, about 1 ng/mL to about 20 ng/mL, about 1 ng/mL to about 30 ng/mL, about 1 ng/mL to about 40 ng/mL, about 1 ng/mL to about 50 ng/mL, about 1 ng/mL to about 100 ng/mL, about 2 ng/mL to about 5 ng/mL, about 2 ng/mL to about 10 ng/mL, about 2 ng/mL to about 20 ng/mL, about 2 ng/mL to about 30 ng/mL, about 2 ng/mL to about 40 ng/mL, about 2 ng/mL to about 50 ng/mL, about 2 ng/mL to about 100 ng/mL, about 5 ng/mL to about ng/mL, about 5 ng/mL to about 20 ng/mL, about 5 ng/mL to about 30 ng/mL, about 5 ng/mL to about 40 ng/mL, about 5 ng/mL to about 50 ng/mL, about 5 ng/mL to about 100 ng/mL, about ng/mL to about 20 ng/mL, about 10 ng/mL to about 30 ng/mL, about 10 ng/mL to about 40 ng/mL, about 10 ng/mL to about 50 ng/mL, about 10 ng/mL to about 100 ng/mL, about 20 ng/mL to about 30 ng/mL, about 20 ng/mL to about 40 ng/mL, about 20 ng/mL to about 50 ng/mL, about 20 ng/mL to about 100 ng/mL, about 30 ng/mL to about 40 ng/mL, about 30 ng/mL to about 50 ng/mL, about 30 ng/mL to about 100 ng/mL, about 40 ng/mL to about 50 ng/mL, about 40 ng/mL to about 100 ng/mL, or about 50 ng/mL to about 100 ng/mL. In some embodiments, spheroids are treated with a concentration of PDGF of about 1 ng/mL, about 2 ng/mL, about 5 ng/mL, about 10 ng/mL, about 20 ng/mL, about 30 ng/mL, about 40 ng/mL, about 50 ng/mL, or about 100 ng/mL. In some embodiments, spheroids are treated with a concentration of PDGF of at least about 1 ng/mL, about 2 ng/mL, about 5 ng/mL, about 10 ng/mL, about 20 ng/mL, about 30 ng/mL, about 40 ng/mL, or about 50 ng/mL. In some embodiments, spheroids are treated with a concentration of PDGF of at most about 2 ng/mL, about 5 ng/mL, about 10 ng/mL, about 20 ng/mL, about 30 ng/mL, about 40 ng/mL, about 50 ng/mL, or about 100 ng/mL. Spheroids may be treated for about 1, 2, 3, 4, 5, 6, 7, 8, 9, 10, 11, 12, 13, 14, or more days with PDGF.

In some embodiments, a composition described comprises one type or a plurality of types of spheroids, wherein the spheroids are treated with a concentration of vascular endothelial growth factor (VEGF). In some embodiments, spheroids are treated with a concentration of PDGF of about 0.5 ng/mL to about 20 ng/mL. In some embodiments, spheroids are treated with a concentration of PDGF of about 0.5 ng/mL to about 1 ng/mL, about 0.5 ng/mL to about 2 ng/mL, about 0.5 ng/mL to about 3 ng/mL, about 0.5 ng/mL to about 4 ng/mL, about 0.5 ng/mL to about 5 ng/mL, about 0.5 ng/mL to about 6 ng/mL, about 0.5 ng/mL to about 7 ng/mL, about ng/mL to about 8 ng/mL, about 0.5 ng/mL to about 9 ng/mL, about 0.5 ng/mL to about 10 ng/mL, about 0.5 ng/mL to about 20 ng/mL, about 1 ng/mL to about 2 ng/mL, about 1 ng/mL to about 3 ng/mL, about 1 ng/mL to about 4 ng/mL, about 1 ng/mL to about 5 ng/mL, about 1 ng/mL to about 6 ng/mL, about 1 ng/mL to about 7 ng/mL, about 1 ng/mL to about 8 ng/mL, about 1 ng/mL to about 9 ng/mL, about 1 ng/mL to about 10 ng/mL, about 1 ng/mL to about 20 ng/mL, about 2 ng/mL to about 3 ng/mL, about 2 ng/mL to about 4 ng/mL, about 2 ng/mL to about 5 ng/mL, about 2 ng/mL to about 6 ng/mL, about 2 ng/mL to about 7 ng/mL, about 2 ng/mL to about 8 ng/mL, about 2 ng/mL to about 9 ng/mL, about 2 ng/mL to about 10 ng/mL, about 2 ng/mL to about 20 ng/mL, about 3 ng/mL to about 4 ng/mL, about 3 ng/mL to about 5 ng/mL, about 3 ng/mL to about 6 ng/mL, about 3 ng/mL to about 7 ng/mL, about 3 ng/mL to about 8 ng/mL, about 3 ng/mL to about 9 ng/mL, about 3 ng/mL to about 10 ng/mL, about 3 ng/mL to about 20 ng/mL, about 4 ng/mL to about 5 ng/mL, about 4 ng/mL to about 6 ng/mL, about 4 ng/mL to about 7 ng/mL, about 4 ng/mL to about 8 ng/mL, about 4 ng/mL to about 9 ng/mL, about 4 ng/mL to about 10 ng/mL, about 4 ng/mL to about 20 ng/mL, about 5 ng/mL to about 6 ng/mL, about 5 ng/mL to about 7 ng/mL, about 5 ng/mL to about 8 ng/mL, about 5 ng/mL to about 9 ng/mL, about 5 ng/mL to about 10 ng/mL, about 5 ng/mL to about 20 ng/mL, about 6 ng/mL to about 7 ng/mL, about 6 ng/mL to about 8 ng/mL, about 6 ng/mL to about 9 ng/mL, about 6 ng/mL to about 10 ng/mL, about 6 ng/mL to about 20 ng/mL, about 7 ng/mL to about 8 ng/mL, about 7 ng/mL to about 9 ng/mL, about 7 ng/mL to about 10 ng/mL, about 7 ng/mL to about 20 ng/mL, about 8 ng/mL to about 9 ng/mL, about 8 ng/mL to about 10 ng/mL, about 8 ng/mL to about 20 ng/mL, about 9 ng/mL to about 10 ng/mL, about 9 ng/mL to about 20 ng/mL, or about 10 ng/mL to about 20 ng/mL. In some embodiments, spheroids are treated with a concentration of PDGF of about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, or about 20 ng/mL. In some embodiments, spheroids are treated with a concentration of PDGF of at least about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, or about 10 ng/mL. In some embodiments, spheroids are treated with a concentration of PDGF of at most about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, or about 20 ng/mL. Spheroids may be treated for about 1, 2, 3, 4, 5, 6, 7, 8, 9, 10, 11, 12, 13, 14, or more days with VEGF.

The methods described herein can further comprise culturing the spheroids or the cells or cell precursors with a non-growth factor supplement. In certain embodiments, the non-growth factor supplement is one or more of Insulin, Transferrin, Sodium Selenite, BSA, and Ethanolamine.

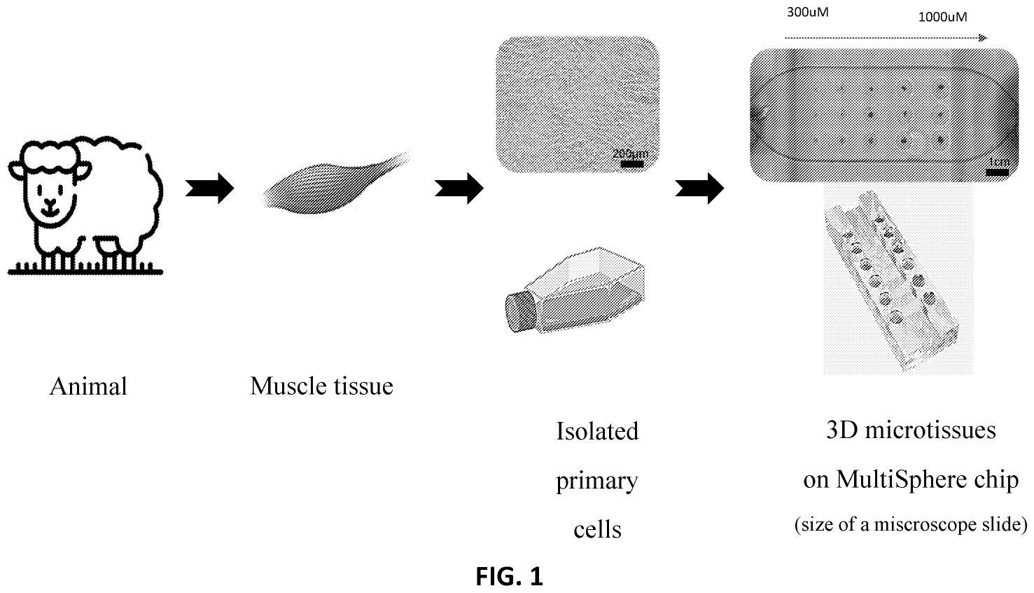

FIG. 1 depicts a schematic diagram of a workflow for cell isolation and cultivation from a non-human animal according to at least some embodiments disclosed herein.

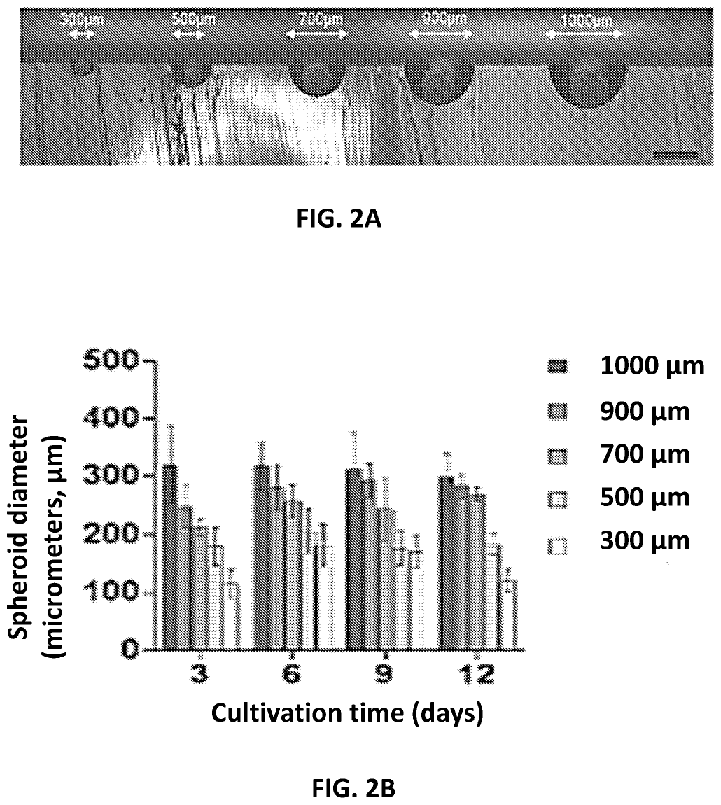

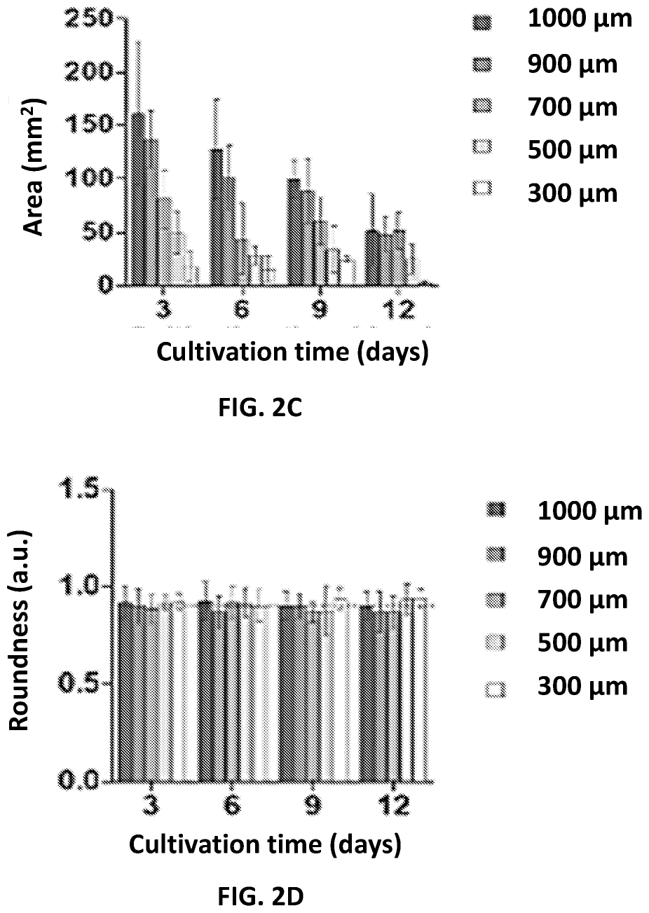

FIG. 2A depicts a cross-section of the microfluidics chip, generating different spheroid sizes. FIG. 2B, FIG. 2C, and FIG. 2D depict morphological parameters of primary animal cells (p4) on the microfluidics chip. More specifically, FIG. 2B depicts a graph having an x-axis representing cultivation time (in days) and a y-axis representing spheroid diameter (in μm). FIG. 2C depicts a graph having an x-axis representing cultivation time (in days) and a y-axis representing area (in mm2), showcasing microtissue area. FIG. 2D depicts a graph having an x-axis representing cultivation time (in days) and a y-axis representing roundness (in a.u.), showcasing a roundness factor for each well diameter, where n=6±SD. As such, the user is capable of selecting spheroids based on area, diameter, and roundness, which will ultimately impact efficiency of scalability described herein.

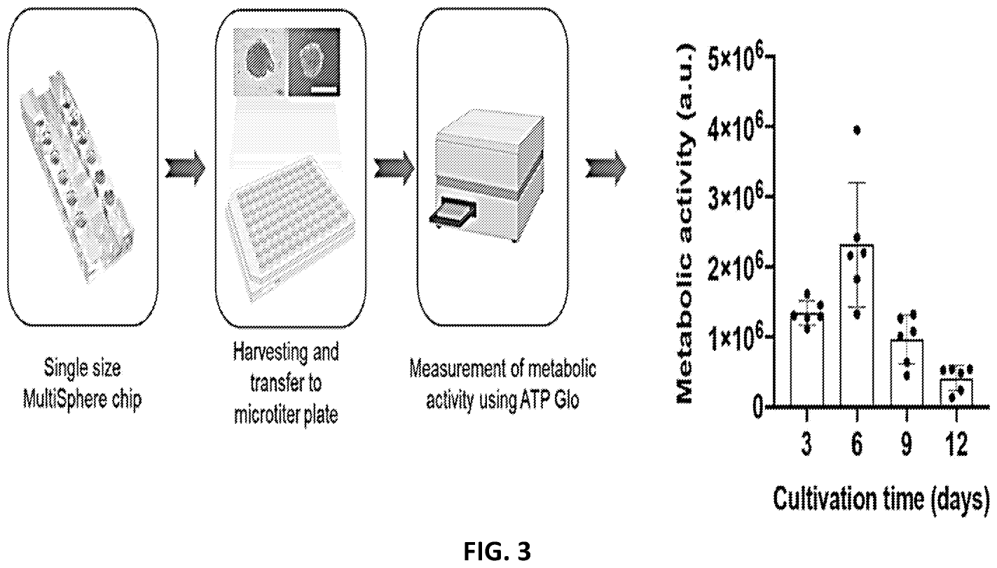

FIG. 3 depicts a method, as well as a graph. The graph includes an x-axis representing cultivation time (in days) and a y-axis representing metabolic activity (in a.u.). FIG. 3 depicts primary animal 3D microtissues (p4) of one size (200 μm in diameter) cultivated in the microfluidics chip and harvested after respective time points of 3, 6, 9 and 12 days post-seeding. The scale bar for FIG. 3 is 200 μm. Metabolic activity for FIG. 3 was measured in a standard plate reader (Enspire2300, PerkinElmer) using a luminescent ATP assay, with n=6±SD. Such allows the user options to select spheroid sizes that exhibit desired metabolic output for downstream applications and scale-up.

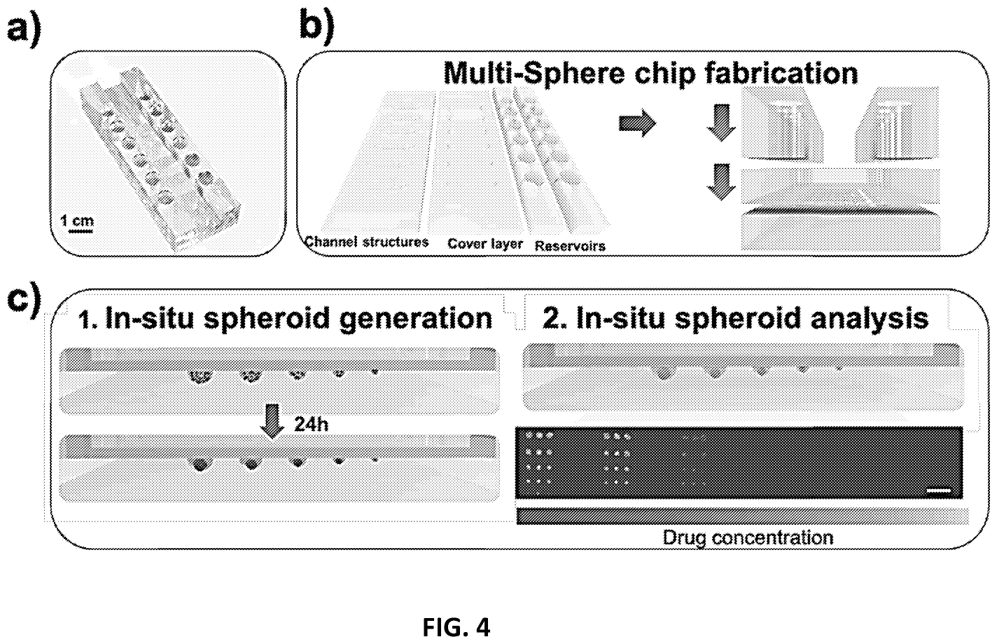

FIG. 4A depicts a cutaway rendering of the microfluidics chip, showing six microfluidic channels containing 15 spheroids, and a pair of media reservoirs for each channel which can be addressed by multi-channel pipettes. FIG. 4B depicts the platform that comprises the microfluidic channel structure and a cover layers consisting of twelve connecting holes, which are fluidically coupled to the reservoir layer ensuring continuous media perfusion. FIG. 4C depicts a workflow of microfluidics chip spheroid generation within 24 hours and in-situ analysis of cellular health under various treatment conditions, with the scale bar of 5 mm.

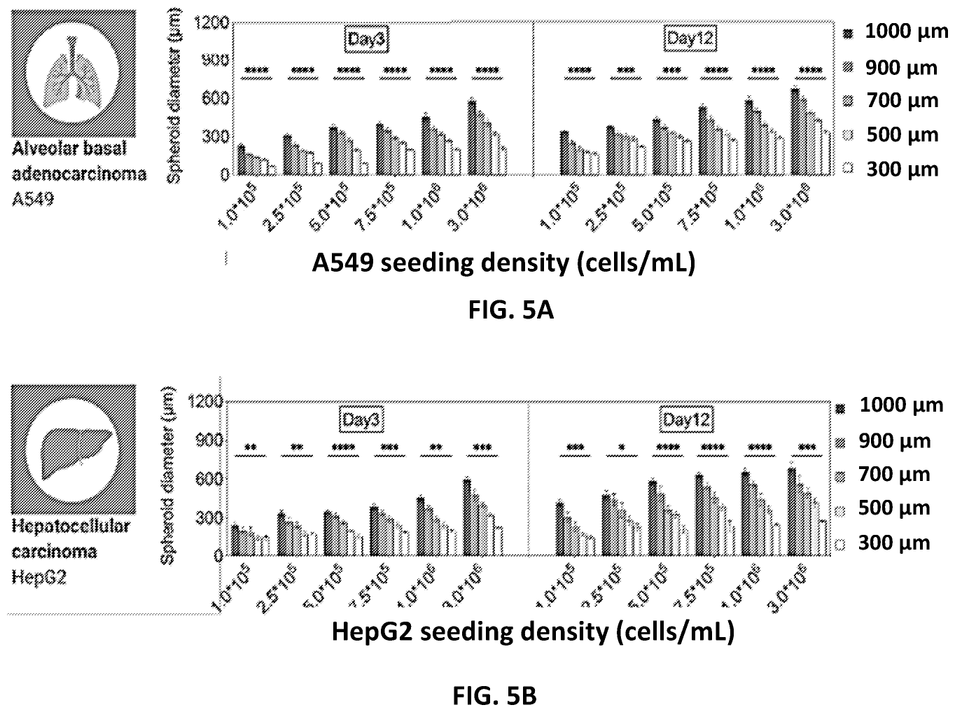

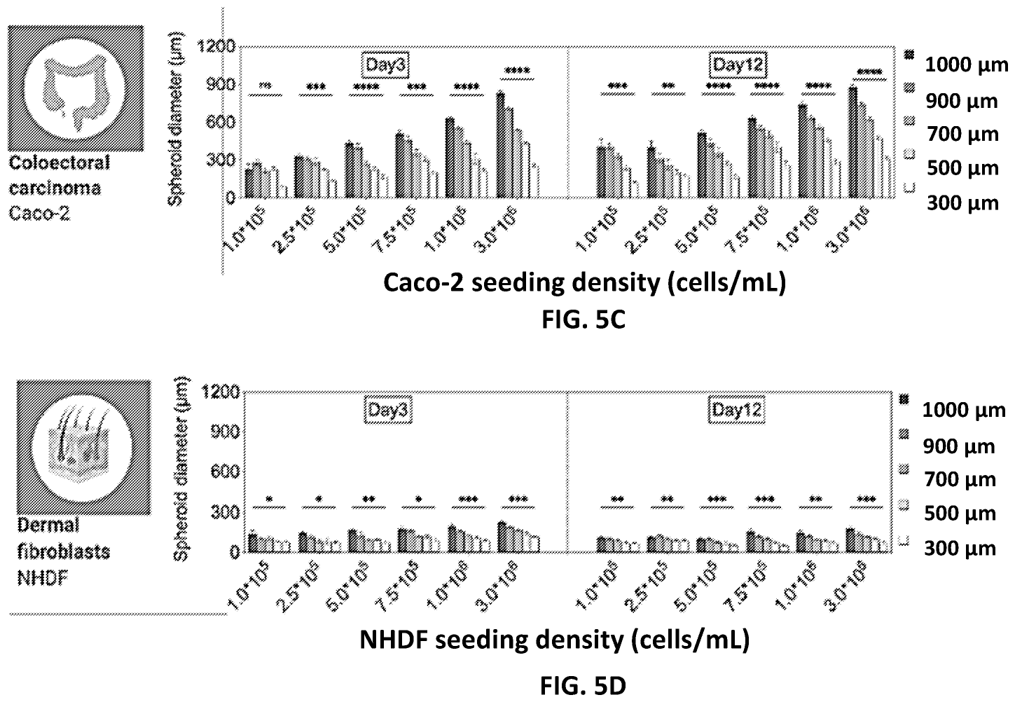

FIG. 5A to FIG. 5D depict graphs having an x-axis of seeding density (in cells/mL) and a y-axis of spheroid diameter (in μm). More specifically, FIG. 5A depicts spheroid diameter change of 3D spheroids of A549 cells. A549 cells are adenocarcinomic human alveolar basal epithelial cells. FIG. 5B depicts spheroid diameter change of 3D spheroids of HepG2 cells. HepG2 cells are a human liver cancer cell line. FIG. 5C depicts spheroid diameter change of 3D spheroids of CacO-2 cells. Caco-2 is an immortalized cell line of human colorectal adenocarcinoma cells. In culture, Caco-2 cells spontaneously differentiate into a heterogeneous mixture of intestinal epithelial cells. FIG. 5D depicts normal human dermal fibroblasts (NHDF) cells at five different seeding densities for a cultivation period of 12 days, with n=9±SD. Statistical analysis was performed using mixed-effects analysis (*p<0.0332, **p<0.0021, ***p<0002, and ****p<0.0001).

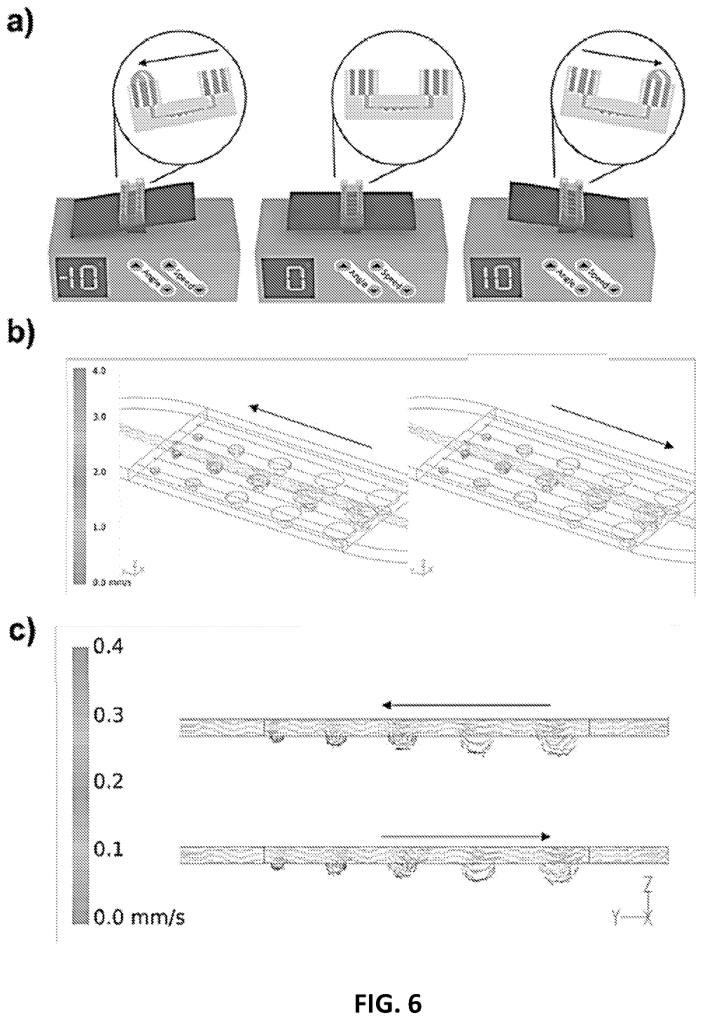

FIG. 6A depicts tilting schemes of the microfluidics chip system by gravity-driven flow. FIG. 6B and FIG. 6C depict bidirectional fluid profiles of flow velocities (mm/s) during tilting of the microfluidics chip, ensuring a continuous media perfusion of spheroids in each well during cultivation.

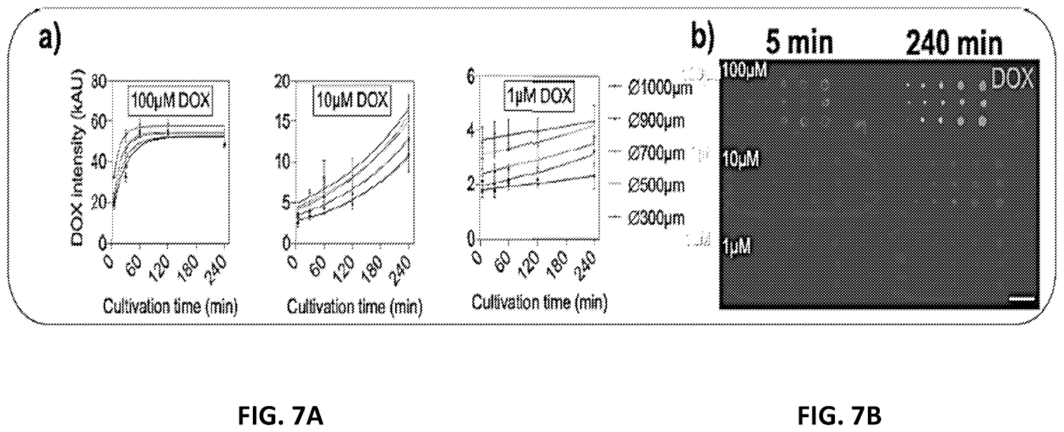

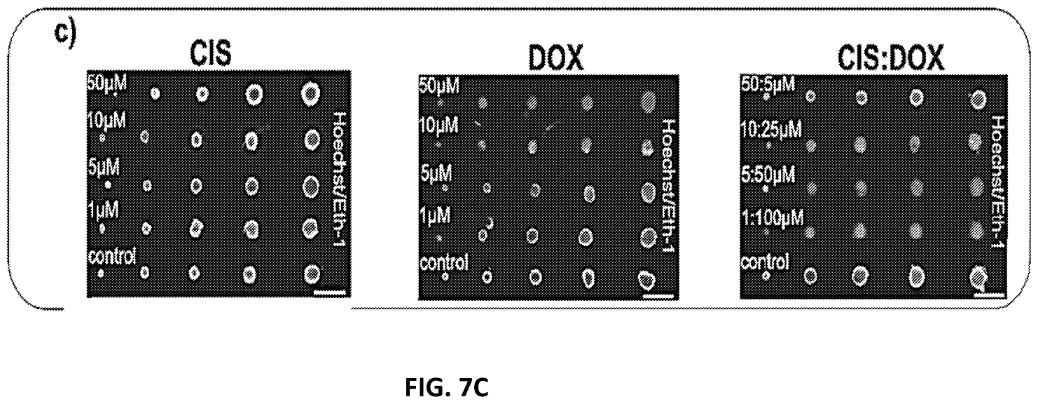

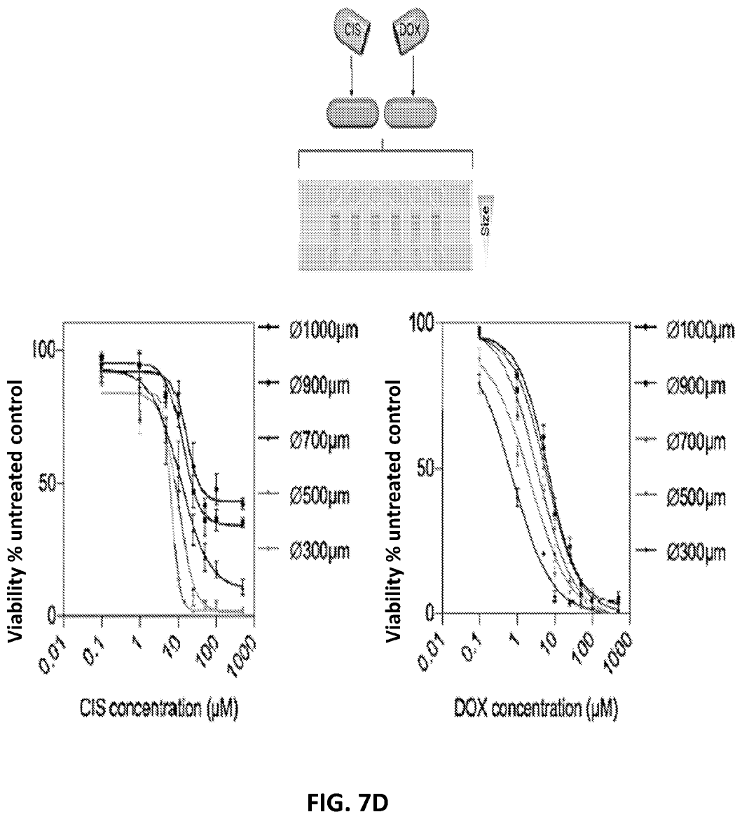

FIG. 7A depicts a graph having an x-axis of cultivation time (in minutes) and a y-axis of Doxorubicin (DOX) intensity (in kAU). More specifically, FIG. 7A depicts on-chip monitoring of microtissue penetration of 100 μM, 10 μM and 1 μM DOX in A540 spheroids of different sizes over a cultivation period of 4 hours, with n=6±SD. A549 cells are adenocarcinomic human alveolar basal epithelial cells. FIG. 7B depicts micrographs of different-sized A549 spheroids in the microfluidics chip treated with Cisplatin (CIS), Doxorubicin (DOX) and a combination of both (CIS:DOX) for 24 hours to screen drug toxicity by staining with cell nuclei (Hoechst; blue) and dead cells (Ethidium homodimer-1; red).

FIG. 7C depicts dose response relations of CIS and DOX treated A549 spheroids of different sizes (generated in 1000 μm, 900 μm, 700 μm, 500 μm and 300 μm microwells) in the microfluidics chip device for 24 hours, with n=6±SD. FIG. 7D depicts a first graph having an x-axis of CIS concentration (in μm) and a y-axis of viability (measured as a % to the untreated control) and a second graph having an x-axis of DOX concentration (in μm) and a y-axis of viability (measured as a % to the untreated control). More specifically, FIG. 7D depicts a statistical analysis of respective CIS and DOX concentrations performed using mixed-effects model. (*p<0.0332, **p<0.0021, ***p<0002, and ****p<0.0001).

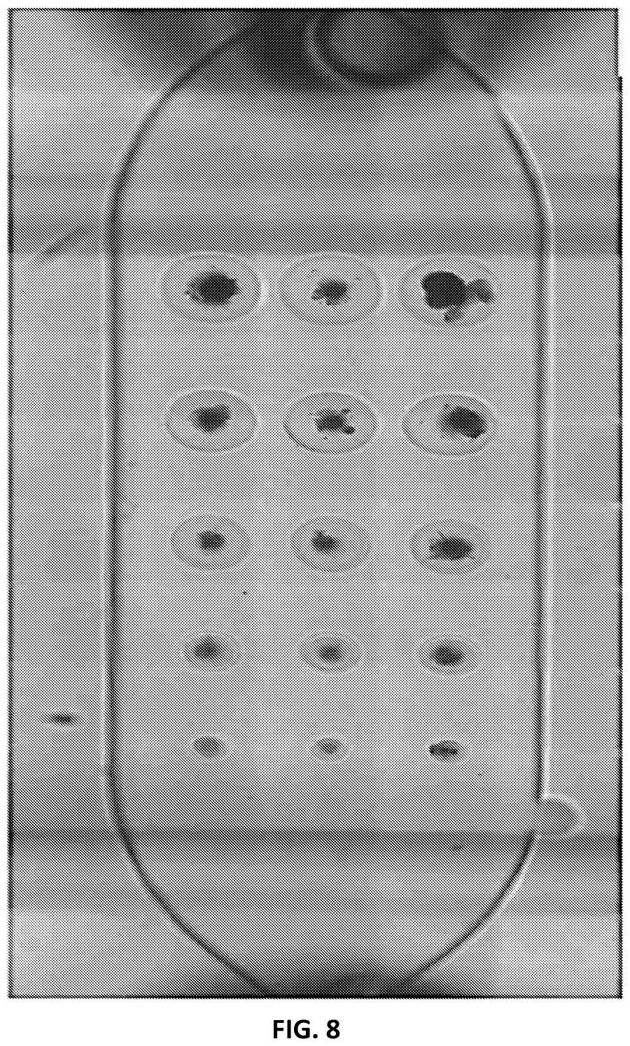

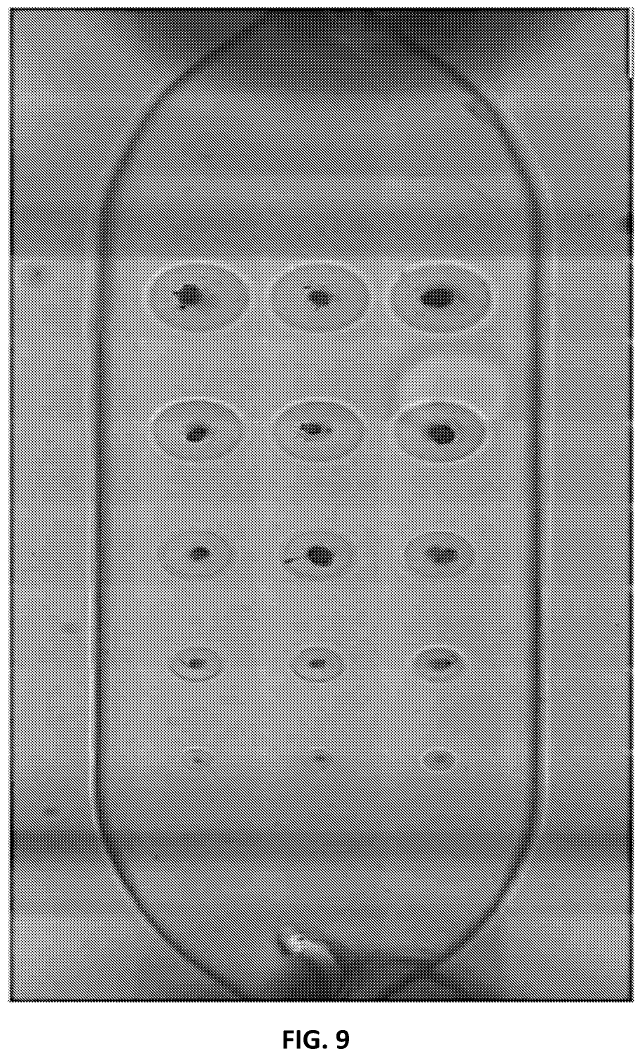

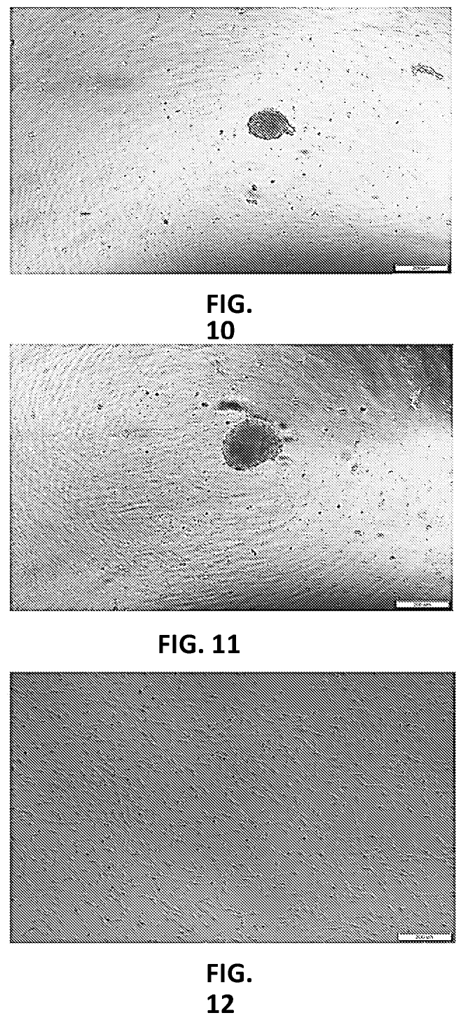

FIG. 8 depicts an image of spheroid seeding at day three and FIG. 9 depicts an image of spheroid seeing at day twelve, with a seeding density of 3×106/mL. FIG. 10 depicts an image of three-days post-harvest, FIG. 11 depicts an image of six days post-harvest, and FIG. 12 depicts an image of sheep muscle (p5).

FIG. 14 depicts an image of sheep muscle spheroids on-chip showing viability staining. Characterization of sheep muscle spheroids on the chip included staining for viability, density, protein expression, differentiation oxygen diffusion, and/or nutrient uptake. Further testing and characterization can be accomplished after harvesting the spheroids.

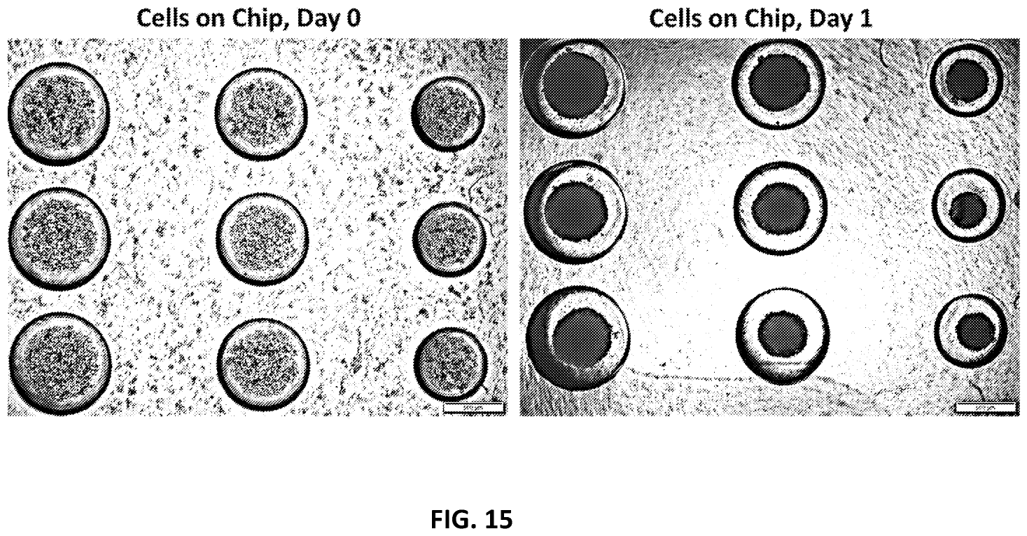

FIG. 15 depicts an image of mammalian embryonic stem cells cultured on-chip as spheroids at day zero and at day one. The variety of cell types that can be cultured as spheroids on the chip includes embryonic stem cells. Cells cultured on the chip as spheroids allow optimized screening of multiple media conditions for different animal cell lines of all species, selection of optimized spheroids based on size, metabolic activity, desired differentiation into muscle, fat, or other cells, or any combination thereof.

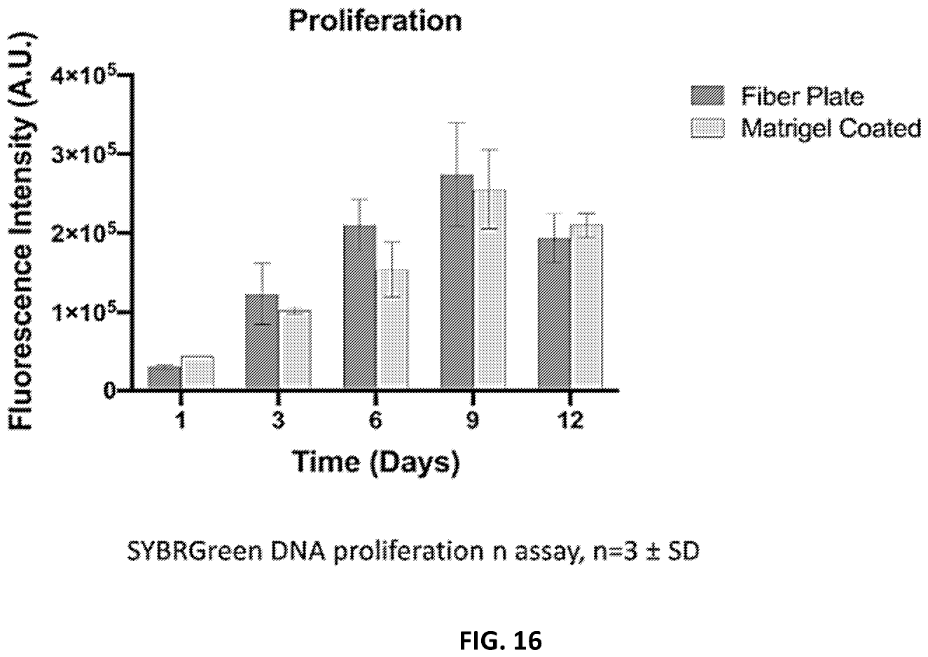

FIG. 16 depicts a graph having an x-axis of cell culture time (in days) and a y-axis of fluorescence intensity (in A.U.). Cells cultured for 12 days on fiber and 10% Matrigel™ coated plates exhibited different DNA proliferation profiles as shown by fluorescence intensity of stained live cells.

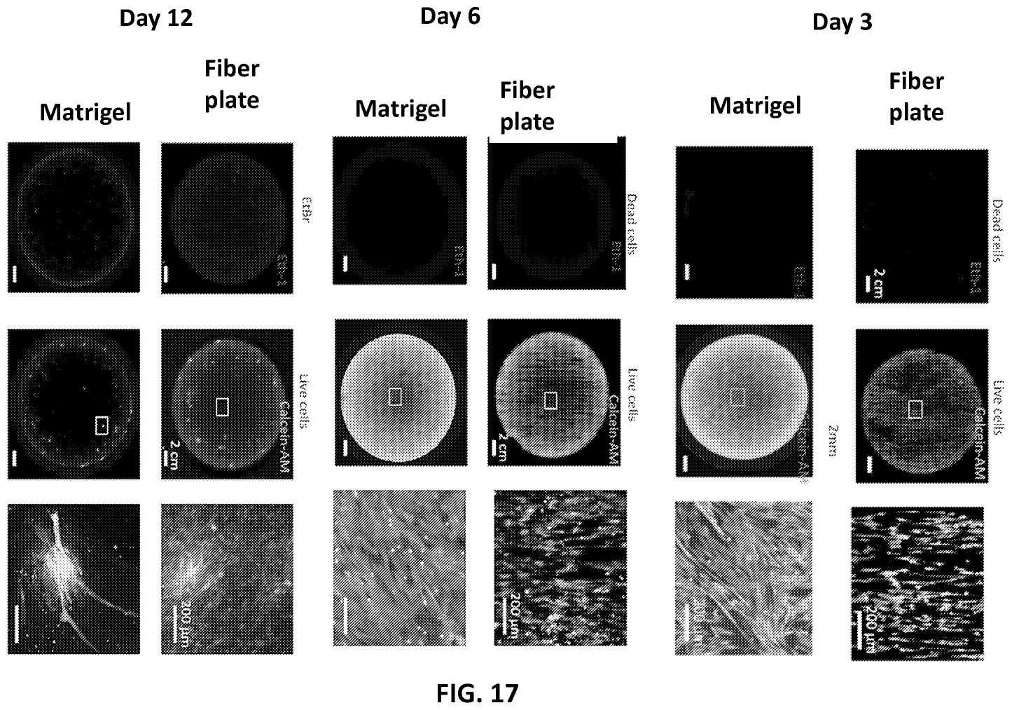

FIG. 17A to FIG. 17C depicts cells cultured for 12 days on fiber and 10% Matrigel™ coated plates exhibited different DNA proliferation profiles as shown by fluorescence intensity of stained live cells. More specifically, FIG. 17A shows cells cultured for 3 days, FIG. 17B shows cells cultured for 6 days, and FIG. 17C shows cells cultured for 12 days.

FIG. 18A to FIG. 18C depicts the metabolic activity (in relative light units) of 3D tissues of three sizes (300 μm, 500 μm, 700 μm in diameter) cultured for 3 and 5 days following growth factor treatment with fibroblast growth factor (FGF), hepatocyte growth factor (HGF), and insulin-like growth factor (IGF). More specifically, FIG. 18A shows data for 3D tissues 300 μm in diameter, FIG. 18B shows data for 3D tissues 500 μm in diameter, and FIG. 18C shows data for 3D tissues 700 μm in diameter.

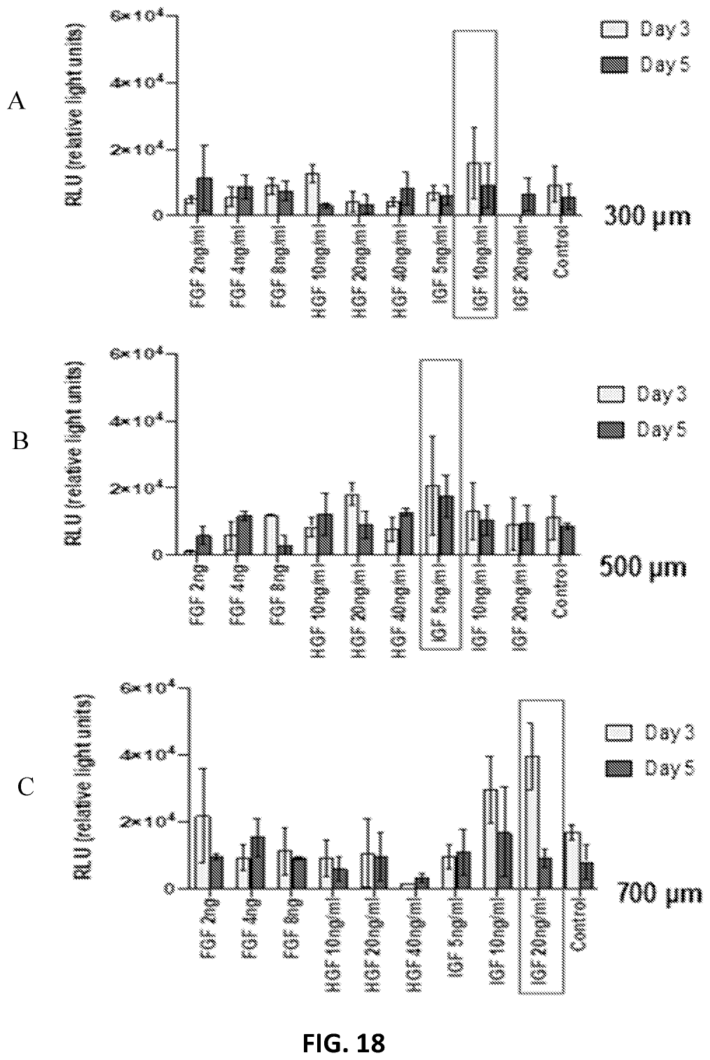

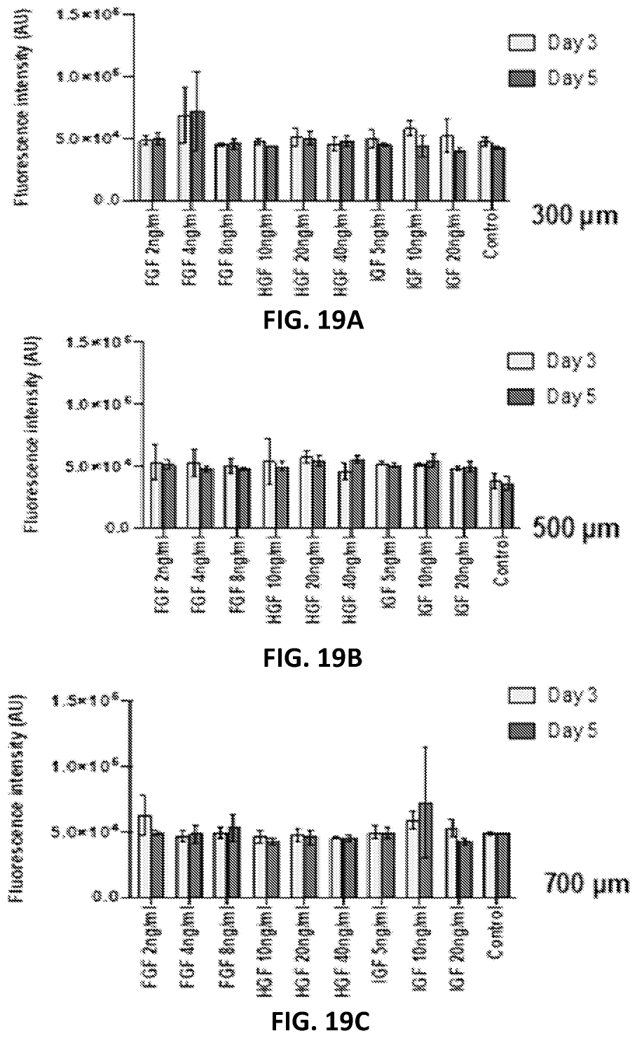

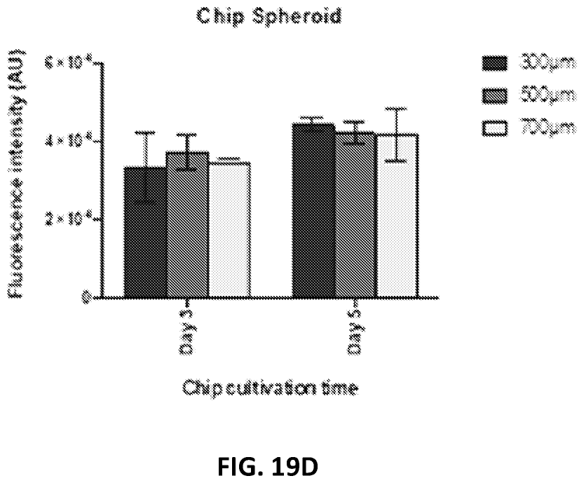

FIG. 19A to FIG. 19D depicts the metabolic activity (in fluorescence intensity) of 3D tissues of three sizes (300 μm, 500 μm, 700 μm in diameter) cultured for 3 and 5 days following growth factor treatment with fibroblast growth factor (FGF), hepatocyte growth factor (HGF), and insulin-like growth factor (IGF). More specifically, FIG. 19A shows data for 3D tissues 300 μm in diameter, FIG. 19B shows data for 3D tissues 500 μm in diameter, FIG. 19C shows data for 3D tissues 700 μm in diameter, and FIG. 19D shows data for the control group.

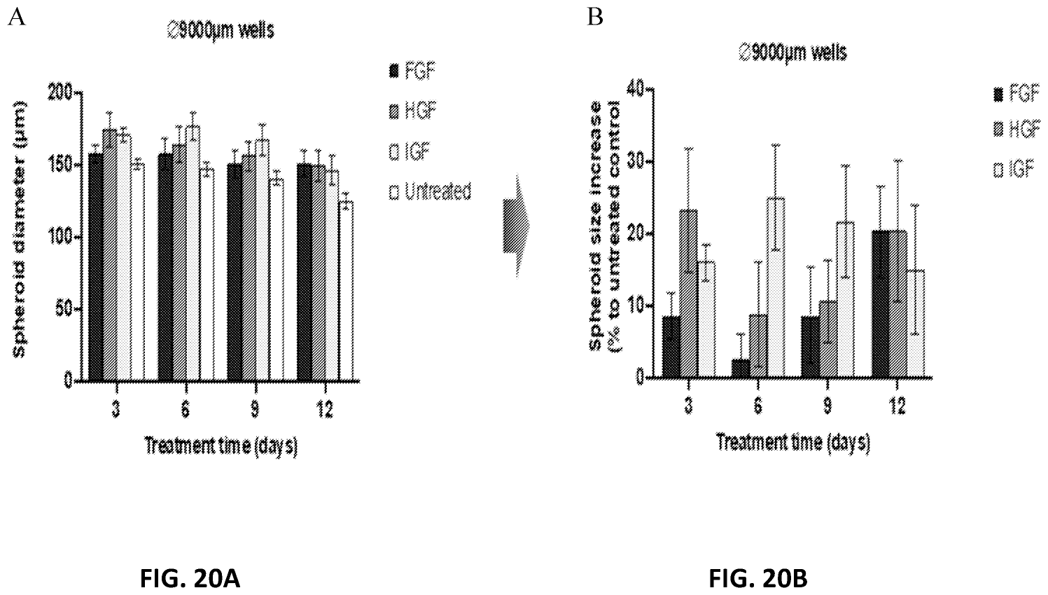

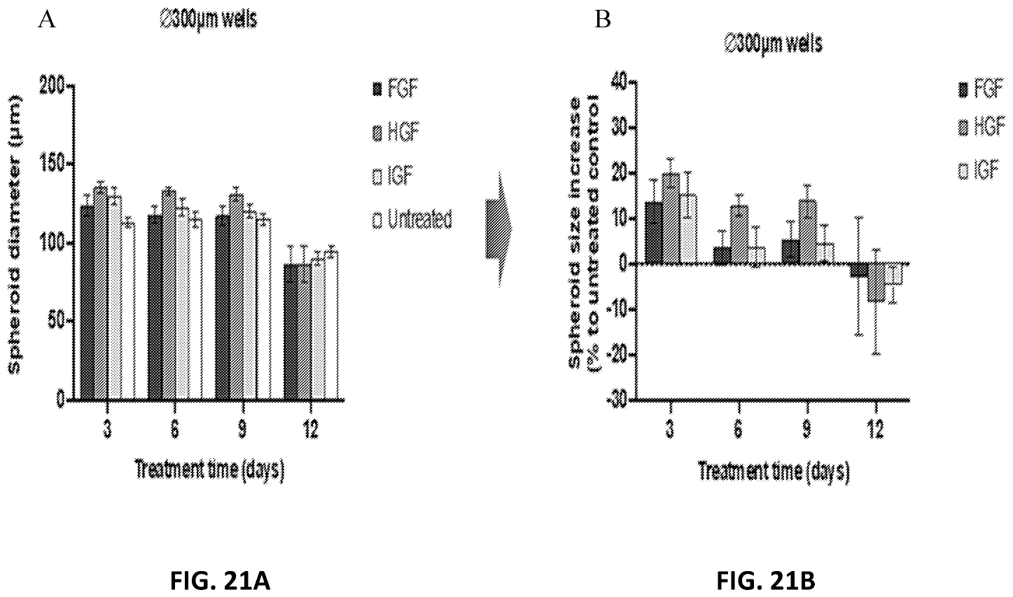

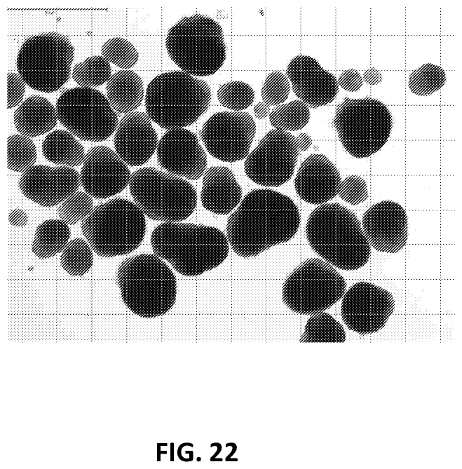

FIG. 20A to FIG. 20B depicts spheroid diameter of cells cultured in wells measuring 900 μm in diameter for 12 days following growth factor treatment with fibroblast growth factor (FGF), hepatocyte growth factor (HGF), and insulin-like growth factor (IGF). More specifically, FIG. 20A shows depicts spheroid diameter in and FIG. 20B depicts spheroid diameter as a percentage of size increase as compared to control. FIG. 21A to FIG. 21B depicts spheroid diameter of cells cultured in wells measuring 300 μm in diameter for 12 days following growth factor treatment with fibroblast growth factor (FGF), hepatocyte growth factor (HGF), and insulin-like growth factor (IGF). More specifically, FIG. 21A depicts spheroid diameter in and FIG. 21B depicts spheroid diameter as a percentage of size increase as compared to control.

Method

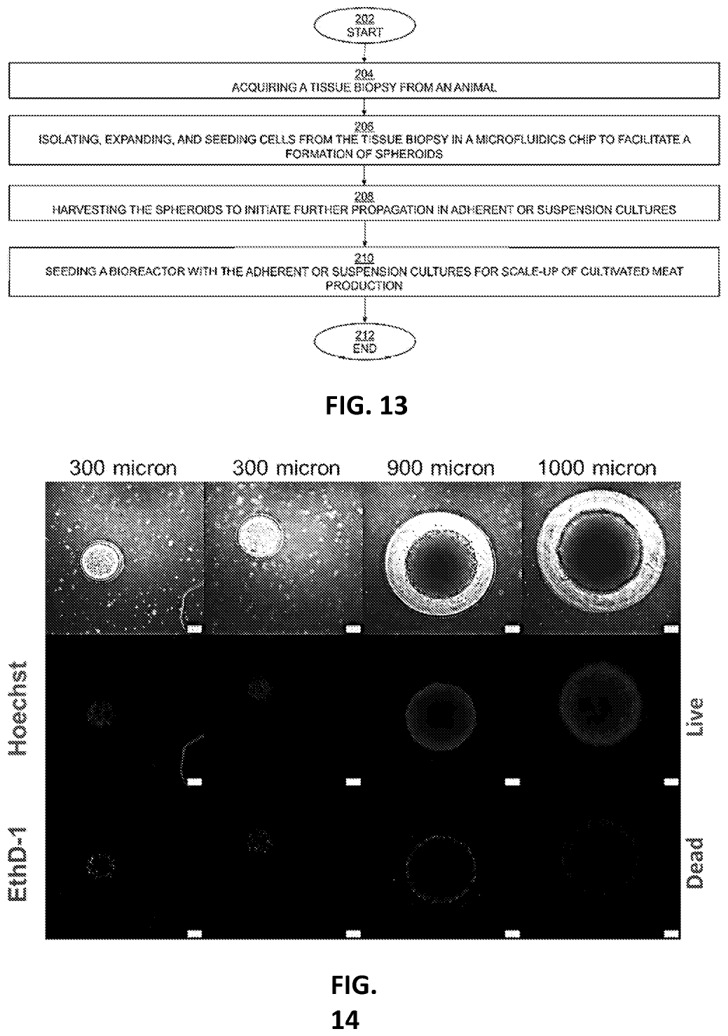

The present invention describes a method. The method includes numerous process steps. The method of FIG. 13 may begin at a process step 202. A process step 204 of FIG. 13 may follow the process step 202 and may include acquiring a tissue biopsy from an animal. The animal may be a cow, a pig, a chicken, a fish, a sheep, a bison, a wagyu cattle, a duck, a goose, an elk, a deer, or an ostrich, among other examples not explicitly listed herein. In other examples, the process step 202 and may include acquiring the tissue biopsy from embryonic stem cells. The tissue biopsy may be acquired by any means known to those having ordinary skill in the art.

A process step 206 of FIG. 13 follows the process step 204 and includes: isolating, expanding, and seeding cells from the tissue biopsy in a microfluidics chip to facilitate a formation of spheroids. The isolation of the animal or embryonic stem cells may occur under typical culture conditions (e.g., 37° C. and 5% CO2). The spheroids may be in a range of approximately 10 μm to approximately 10 mm.

The spheroids may include a singular cell type. In another example, the spheroids may include a mixture of cell types. For example, the mixture of cell types may include muscle cells, liver cells, endothelial cells, etc. The spheroids may also comprise a varying ratio of muscle to fat. For examples, the spheroids may comprise a ratio of muscle to fat of 30:70 or 50:50, among other examples not explicitly listed herein.

A process step 208 of FIG. 13 follows the process step 206 and includes: harvesting the spheroids to initiate further propagation in adherent or suspension cultures. A process step 210 follows the process step 208 and includes: seeding a bioreactor with the adherent or suspension cultures for scale-up of cultivated meat production. In the bioreactor, the tissue growth will expand from the spheroids. In other examples, the bioreactor may be used to scale-up growth with high cell density. It should be appreciated that additional cells may be added during the scale-up process. In some examples, the spheroids comprise scaffolding. In other examples, the spheroids do not comprise scaffolding. Moreover, in some examples, the process step 210 may include transferring the spheroids to a flask or a bag instead of a bioreactor.

Moreover, the method may include optionally varying a composition of the spheroids to modify a density of the spheroids and/or properties of a meat product. Such properties may include: a texture of the product, a taste of the product, and/or a mouthfeel of the product, among others. A process step 212 of FIG. 13 follows the process step 210 and concludes the method of FIG. 13. It should be appreciated that a schematic diagram of a workflow for cell isolation may be seen in FIG. 1.

As such, the method of FIG. 13 and the instant invention uses organoids and/or spheroids for the purpose of cultivated meat or cultivated meat products, which has not been done previously. The present invention allows for the growth of cells to high densities on a microfluidics chip, which can then be used as seed stock in downstream applications for cultivated meat/cultivated meat products. The ability to propagate organoids on the microfluidics chip allows for low cost high throughput screening for media, cellular differentiation, co-cultured systems, metabolic activity, and suspension adaptation features in the context of cultivated meat. The method wherein the spheroid ratios of cartilage muscle fat are equivalent to the composition of sirloin, ribeye, short loin, flank, plate, brisket, rib, round, shank, filet, leg, belly, ham, shoulder, loin, chops, wing, thigh, drumstick, breast, or any combination thereof.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### Specific Embodiments

Certain specific embodiments are envisioned which include:
1. A method comprising:
   a. acquiring cells from a non-human animal source;
   b. expanding cells from the non-human animal source in a culture vessel, on a plate or on a microfluidics chip to facilitate formation of spheroids or organoids;
   c. harvesting the spheroids to initiate further propagation in adherent or suspension cultures;
   d. and optionally seeding a bioreactor with the adherent or suspension cultures for scale-up of cultivated meat production.
2. The method of embodiment 1, wherein acquiring cells from a non-human animal source comprises acquiring cells from a tissue biopsy, an immortalized cell line, stem cells, precursor cells, embryonic cells, bone marrow, or any combination thereof.
3. The method of embodiment 1, wherein the method includes screening cells, spheroids, or organoids for a physical attribute.
4. The method of embodiment 1, wherein the spheroids possess a diameter from about 10 μm to about 10 mm.
5. The method of embodiment 1, wherein the spheroids comprise a singular cell type.
6. The method of embodiment 1, wherein the spheroids comprise a mixture of cell types.
7. The method of embodiment 1, wherein the spheroids comprise stem cells such as embryonic stem cells, induced pluripotent stem cells, mesenchymal stem cells, and/or hematopoietic stem cells.
8. The method of embodiment 1, wherein the spheroids comprise scaffolding.
9. The method of embodiment 1, wherein the spheroids do not comprise scaffolding.
10. The method of embodiment 1, further comprising: varying a composition of the spheroids to modify a density of the spheroids and/or properties of a meat product.
11. The method of embodiment 10, wherein the properties are selected from the group consisting of: a texture of the product, a taste of the product, and a mouthfeel of the product.
12. The method of embodiment 1, wherein the animal is selected from the group consisting of: a cow, a pig, a chicken, a fish, a sheep, a bison, a wagyu cattle, a duck, a goose, an elk, a deer, a Berkshire pig, a Kurobuta pig, an Iberian pig, and an ostrich.
13. The method of embodiment 1, wherein the spheroids comprise a specific ratio of muscle to fat.
14. The method of any one of embodiments 1 to 13, wherein the spheroids are cultured in a heterologous extracellular matrix.
15. The method of any one of embodiments 1 to 13, wherein the spheroids are cultured in a heterologous extracellular matrix comprising from about 5% to about 15% or from about 6% to about 14% gelatinous protein mixture secreted by heterologous cells.
16. The method of any one of embodiments 1 to 13, wherein the spheroids are cultured in a heterologous extracellular matrix comprising a hydrogel or laminin.
17. The method of any one of embodiments 1 to 16, wherein the spheroids are treated with a recombinant growth factor selected from the group consisting of fibroblast growth factor (FGF), hepatocyte growth factor (HGF), and insulin-like growth factor (IGF).
18. The method of any one of embodiments 1 to 16, wherein the spheroids are treated with two recombinant growth factors selected from the group consisting of fibroblast growth factor (FGF), hepatocyte growth factor (HGF), and insulin-like growth factor (IGF).
19. The method of any one of embodiments 1 to 16, wherein the spheroids are treated with all of the recombinant growth factors selected from the group consisting of fibroblast growth factor (FGF), hepatocyte growth factor (HGF), and insulin-like growth factor (IGF).
20. The method of any one of embodiments 1 to 19, wherein the spheroids are treated with fibroblast growth factor (FGF).
21. The method of any one of embodiments 1 to 19, wherein the spheroids are treated with hepatocyte growth factor (HGF).
22. The method of any one of embodiments 1 to 19, wherein the spheroids are treated with insulin-like growth factor (IGF).
23. The method of any one of embodiments 1 to 22, wherein the spheroids are treated with about 1 to about 20 ng/mL fibroblast growth factor (FGF).
24. The method of any one of embodiments 1 to 23, wherein the spheroids are treated with about 5 to about 100 ng/mL hepatocyte growth factor (HGF).
25. The method of any one of embodiments 1 to 24, wherein the spheroids are treated with about 1 to about 50 ng/mL insulin-like growth factor (IGF).
26. A composition comprising a first type of spheroid comprising a first plurality of non-human animal cells, wherein said cells comprise one or more interactions selected form the list consisting of cell-to-cell, cell- to extracellular matrix (ECM) interactions, and a combination thereof, wherein the composition optionally further comprises a sterile medium free of animal serum.
27. The composition of embodiment 26, wherein the spheroid comprises two or more cell types.
28. The composition of embodiment 26 or 27, further comprising a second type of spheroid comprising a second plurality of non-human animal cells, wherein the second plurality of cells is of a different tissue type from the plurality of non-human animal cells of the first spheroid.
29. The composition of any one of embodiments 26 to 28, wherein the first and second type of spheroids form a three-dimensional structure wherein about 50% to about 90% of the first or second spheroids are not in contact with an exogenous support or scaffold.
30. The composition of any one of embodiments 26 to 29, wherein the first and second type of spheroids form a three-dimensional structure wherein about 1% to about 49% of the first or second spheroids are not in contact with an exogenous support or scaffold.
31. The composition of embodiment 30, wherein the first type of spheroid has a first average diameter that is different from a second average diameter of the second spheroid.
32. The composition of any one of embodiments 28 to 31, further comprising a third type of spheroid comprising a third plurality of cells, wherein the third plurality of cells is derived from a different tissue type from the first or second spheroid, optionally wherein the third spheroid has a third average diameter that is different from a first and/or a second average diameter.
33. The composition of any one of embodiments 26 to 31, wherein the first plurality of non-human animal cells is selected from a group consisting of stem cells, precursor cells, or differentiated cells comprising muscle cells, connective tissue cells, fat cells, cartilage cells, blood cells, and combinations thereof.
34. The composition of any one of embodiments 26 to 33, wherein the second plurality of non-human animal cells is selected from a group consisting of stem cells, precursor cells, or differentiated cells comprising muscle cells, connective tissue cells, fat cells, cartilage cells, blood cells, and combinations thereof.
35. The composition of any one of embodiments 26 to 34, wherein the third plurality of non-human animal cells is selected from a group consisting of stem cells, precursor cells, or differentiated cells comprising muscle cells, connective tissue cells, fat cells, cartilage cells, blood cells, and combinations thereof.
36. The composition of any one of embodiments 27 to 35, wherein the three-dimensional structure comprises a surface area of 64 to 225 cm².
37. The composition of any one of embodiments 27 to 36, wherein the three-dimensional structure comprises a density of 0.3 to 1.8 g/cm³.
38. The composition of any one of embodiments 26 to 37, wherein the spheroids have a roundness of 0.1-2.
39. The composition of embodiments 31 or 32, wherein the spheroids having different average diameters are of different sizes.
40. The composition of any one of embodiments 28 to 39, wherein the first type of spheroid, second type of spheroid, and/or third type of spheroid possess a diameter from about 10 to about 10000 micrometers.
41. The composition of any one of embodiments 28 to 39, wherein the first type of spheroid, second type of spheroid, and/or third type of spheroid possesses a diameter range from about 50 to about 500 micrometers.
42. The composition of any one of embodiments 28 to 39, wherein the first type of spheroid, second type of spheroid, and/or third type of spheroid possesses a surface area from about 10 to about 40 mm², about 39 to about 100 mm², or about 90 to about 240 mm².
43. The composition of any one of embodiments 28 to 39, wherein the spheroids volumes range from 1 to 5 mm³, 4 to 10 mm³, or 9 to 15 mm³.
44. The composition of any one of embodiments 26 to 43, wherein the composition comprises a specific ratio of muscle cells to fat cells, muscle cells to connective tissue cells, or connective tissue cells to fat cells.
45. The composition of any one of embodiments 26 to 44, wherein the spheroids comprise a specific ratio of muscle cells to fat cells to connective tissue cells.
46. The composition of embodiment 45, further comprising cartilage.
47. The composition of embodiments 42 or 46, wherein the percentage of muscle cells of the composition is from about 50% to about 95%, about 60% to about 95%, or about 75% to about 95%.
48. The composition of embodiments 42 or 46, wherein the percentage of connective tissue cells of the composition is from about 1% to 20%, about 1% to 10%, or about 1% to 5%.
49. The composition of embodiments 42 or 46, wherein the percentage of fat tissue cells of the composition is from about 1% to 50%, about 1% to 30%, about 1% to 20%, or about 1% to 10%.
50. The composition of embodiments 44 or 46, wherein the ratio or percentage of muscle to cartilage cells is from about 0.5% to 3%, about 2% to 5%, or about 4% to 7%.
51. The composition of any one of embodiments 44 to 50, wherein the cells are harvested from a tissue biopsy of a non-human animal.
52. The composition of any one of embodiments 44 to 51, wherein the composition comprises stem cells such as mesenchymal stem cells, and/or hematopoietic stem cells.
53. The composition of any one of embodiments 44 to 52, wherein the non-human animal is selected from the group consisting of: a cow, a pig, a chicken, a fish, a sheep, a bison, a wagyu cattle, a boar, a reptile, an ostrich, a sheep, a goat, a camel, a duck, a goose, an elk, a deer, a Berkshire pig, a Kurobuta pig, an Iberian pig, and a turkey.
54. The composition of embodiments 44 to 53, wherein the spheroid ratios are equivalent to the composition of sirloin, ribeye, short loin, flank, plate, brisket, rib, round, shank, filet, leg, belly, ham, shoulder, loin, chops, wing, thigh, drumstick, breast, or any combination thereof.
55. The composition of any one of embodiments 44 to 54, wherein the spheroid ratios provide a pattern equivalent to marbled meat.
56. The composition of any one of embodiments 51 to 55, wherein the tissue biopsy cells are cultured in a scaffolding or microfluidic chip to facilitate the formation of spheroids.
57. The composition of any one of embodiments 26 to 55, wherein the spheroids are cultured in vitro.
58. The composition of any one of embodiments 26 to 57, comprising a heterologous extracellular matrix.
59. The composition of any one of embodiments 26 to 58, comprising from about 5% to about 15% or from about 6% to about 14% gelatinous protein mixture secreted by Engelbreth-Holm-Swarm mouse sarcoma cells.
60. The composition of any one of embodiments 26 to 59, comprising heterologous extracellular matrix comprising hydrogel.
61. The composition of any one of embodiments 26 to 60, comprising a recombinant growth factor selected from the group consisting of fibroblast growth factor (FGF), hepatocyte growth factor (HGF), and insulin-like growth factor (IGF).
62. The composition of any one of embodiments 26 to 60, comprising fibroblast growth factor (FGF).
63. The composition of any one of embodiments 26 to 60, comprising hepatocyte growth factor (HGF).
64. The composition of any one of embodiments 26 to 60, comprising insulin-like growth factor (IGF).
65. The composition of any one of embodiments 26 to 64, comprising about 1 to about 20 ng/mL fibroblast growth factor (FGF).
66. The composition of any one of embodiments 26 to 65, comprising about 5 to about 100 ng/mL hepatocyte growth factor (HGF).
67. The composition of any one of embodiments 26 to 66, comprising about 1 to about 50 ng/mL insulin-like growth factor (IGF).
68. The composition of any one of embodiments 26 to 67, wherein the plurality of spheroids comprises a volume from about 32 cm³ to about 1800 cm³.

### Examples

The following illustrative examples are representative of embodiments of compositions and methods described herein and are not meant to be limiting in any way.

### Example 1—Viability of Live Cell Spheroid In Vitro

FIG. 2A to FIG. 2D depict live sheep muscle cell spheroids of five sizes (300 μm, 500 μm, 700 μm, 900 μm, 1000 μm in diameter) cultured in vitro for 3 days, 6 days, 9 days, and 12 days on fiber and 10% Matrigel™ coated plates. Spheroid dimensions such as diameter (in μm), area (in mm²), and roundness (in a.u.) were monitored as a factor of time, along with cellular in vitro viability. FIG. 8 and FIG. 9 depict live sheep muscle cell spheroids seeded with a seeding density of 3×10⁶ cells/mL cultured in vitro for 3 days, and 12 days. Prior to seeding sheep muscle cells were grown in cell culture flasks at 37° C. for 7 to 14 days and adherent cells were extracted using trypsin. Sheep muscle cells and spheroids were cultured in Dulbecco's Minimal Essential Medium (DMEM; SigmaAldrich, Austria) supplemented with 5% fetal bovine serum (FBS; Sigma-Aldrich, Austria), 10% Horse Serum, and 1% antibiotic/antimycotic solution (Sigma-Aldrich, Austria).

### Example 2—Tissue-Specific Viability of Live Cell Spheroid In Vitro

FIG. 5A to FIG. 5D depict the in vitro viability of various tissue-specific live human cells (A549 cells, HepG2 cells, CacO-2 cells, and normal human dermal fibroblast (NHDF) cells) seeded at six different seeding densities (1.0×105, 2.5×105, 5.0×105, 7.5×105, 1.0×106, and 3.0×106 cells/mL) cultured for 3 days and 12 days on fiber and 10% Matrigel™ coated plates. Spheroid diameter was monitored as a factor of seeding density for cell spheroids of five sizes (300 μm, 500 μm, 700 μm, 900 μm, 1000 μm in diameter). Spheroid diameter (in μm) was monitored for spheroids of A549 cells (adenocarcinomic human alveolar basal epithelial cells), spheroids of HepG2 cells (human liver cancer cell line), spheroids of CacO-2 cells (human colorectal adenocarcinoma cells that may spontaneously differentiate into heterogeneous mixture of intestinal epithelial cells), and spheroids of NHDF cells (normal human dermal fibroblasts). As depicted, spheroids cultured for 3 days and 12 days demonstrated viability for the different tissue-specific cells at six different seeding densities, as measured by spheroid diameter.

Caco-2 (HTB-37, ATCC, USA), and normal human dermal fibroblasts (NHDF; CRL-2522, ATCC, USA) were cultured with Dulbecco's Minimal Essential Medium (DMEM; SigmaAldrich, Austria) supplemented with 10% fetal bovine serum (FBS; Sigma-Aldrich, Austria) and 1% antibiotic/antimycotic solution (Sigma-Aldrich, Austria). HepG2 cells (EIB-8065, ATCC, USA) were cultivated with supplemented Minimal Essential Medium (MEM; SigmaAldrich, Austria) with 10% FBS and 1% antibiotic/antimycotic solution (Sigma-Aldrich, Austria), and A549 cells (CCL-185, ATCC, USA) were cultivated in Hams F12K Medium (Sigma-Aldrich, Austria) with 10% FBS and 1% antibiotic/antimycotic solution (SigmaAldrich, Austria). Prior to seeding all cell types were cultivated in T75 cell culture flasks at 37° C. in 5% CO2 humidified atmosphere as adherent monolayers. Cells were washed with 1× Phosphate-Saline Buffer (PBS; Sigma-Aldrich) at confluency of 70-80%, and 0.5% Trypsin-EDTA (Sigma-33 Aldrich, Austria) was added for ten minutes to detach cells.

### Example 3—Microtissue Penetration of Live A549 Cell Spheroids Treated with Chemotherapy Drugs In Vitro

FIG. 7A to FIG. 7D depict the in vitro microtissue penetration and viability of live human A549 cell (adenocarcinomic human alveolar basal epithelial cell) spheroids of different sizes (300 μm, 500 μm, 700 μm, 900 μm, 1000 μm in diameter) Cisplatin (CIS) and Doxorubicin (DOX), alone or in combination. Microtissue penetration of live A549 cell spheroids of different sizes (300 μm, 500 μm, 700 μm, 900 μm, 1000 μm in diameter) treated with different concentrations (100 μM, 10 μM and 1 μM) of DOX was monitored for four hours following drug treatment.

A549 cells were seeded at a concentration of 1×106 cells per ml and cultivated for three days under standard cell culture conditions under bi-directional flow (e.g. using a rocking device). Stock solutions of 10 mM cisplatin (Sigma-Aldrich, Austria) in DMSO and 10 mM doxorubicin (Sigma-Aldrich, Austria) in PBS were prepared. Doxorubicin and cisplatin were dissolved in cell culture medium to yield concentrations of 0.5, 1, 5, 10, 25, 50 and 500 μM for the treatment of A549 spheroids. Cell culture medium within the channels of the devices was replaced with drug containing medium and incubated for 24 hours prior to cell death analyses. One channel on a separate device was used as an untreated control. Following the incubation, drug solutions were removed, and 10 μg/ml Hoechst 33342 (Invitrogen, Austria) and 4 μM Ethidium-Homodimer 1 (Invitrogen, Austria) applied. After incubation for 30 minutes, spheroids were imaged using DAPI (ex 390 nm, em 460 nm) and TRITC filters (ex 530 nm, em 645 nm). Raw fluorescence signals were processed and dose-response curves were generated by Sigmoidal-4PL non-linear regression analysis.

Live A549 cell spheroids of different sizes (300 μm, 500 μm, 700 μm, 900 μm, 1000 μm in diameter) were stained with Hoechst; blue and Ethidium homodimer-1; red and treated with different concentrations (100 μM, 10 μM and 1 μM) of CIS, DOX, and CIS:DOX to screen drug toxicity for up to 24 hours following drug treatment. Drug toxicity screening considered stained cell nuclei (Hoechst; blue) and dead cells (Ethidium homodimer-1; red). Viability of live human A549 cell spheroids of different sizes (measured as a % of the untreated control) was calculated as a function of chemotherapy drug treatment concentration (CIS, DOX, and CIS:DOX).

As depicted, viability of live human A549 cell spheroids of all sizes decreased sharply following treatment with different concentrations of DOX.

Monitoring of on-chip A549 spheroid penetration of 100×10-6M, 10×10-6M, and 1×doxorubicin (DOX) over a cultivation period of 4 h, n=6±SD. B) Dose-response relationships of CIS and DOX treated A549 spheroids of different sizes (generated in 1000, 900, 700, 500, and 300 μm microwells) in the spheroid array chip for 24 h using a dye exclusion assay (Hoechst; cell nuclei and ethidium homodimer-1; dead cells), n=4-6±SD. Statistical analysis of respective CIS and DOX concentrations was performed using the mixed-effects model. (*p<0.0332, **p<0.0021, ***p<0.0002, ****p<0.0001). C) Combinatorial on-chip drug screening of CIS and DOX in correlation to untreated controls after 24 h exposure of A549 spheroids of various dimensions, n=3-6±SD. Corresponding fluorescent micrographs of treated different-sized A549 spheroids of CIS:DOX for 24 h to screen drug toxicity by staining cell nuclei (Hoechst; blue) and dead cells (Ethidium homodimer-1; red). Scale bar, 1 mm.

### Example 4—Viability of Live Cell Spheroid in Coated Plates

FIG. 16 illustrates that live cell spheroids remain viable and proliferate (in fluorescence intensity A.U. as measured by SYBER green incorporation) in plates coated with fiber or Matrigel™ for at least 12 days after SYBRGreen treatment. FIG. 17 illustrates that sheep muscle tissue cells cultured for 12 days on fiber and 10% Matrigel™ coated plates exhibited different DNA proliferation profiles as shown by fluorescence intensity of stained live cells. Along with cellular proliferation and viability, the spheroid sizes were monitored.

FIG. 17A to FIG. 17C depicts cells cultured for 12 days on fiber and 10% Matrigel™ coated plates exhibited different DNA proliferation profiles as shown by fluorescence intensity of stained live cells. More specifically, FIG. 17A shows cells cultured for 3 days, FIG. 17B shows cells cultured for 6 days, and FIG. 17C shows cells cultured for 12 days.

### Example 2—Spheroid Metabolic Activity Following Recombinant Growth Factor Treatment

FIG. 18A to FIG. 18C illustrates the metabolic activity (in relative light units) of sheep muscle spheroids of three sizes (300 μm, 500 μm, 700 μm in diameter) cultured in microchip for 3 days and 5 days following recombinant growth factor treatment with fibroblast growth factor (FGF), hepatocyte growth factor (HGF), and insulin-like growth factor (IGF). The metabolic activity of 3D tissues or spheroids varies as a function of recombinant growth factor treatment type and concentration, and as a function of 3D tissue size. The metabolic activity of 3D tissues cultured for 3 days was greater than that of 3D tissues cultured for 5 days for all tissue sizes. As depicted, the highest metabolic activity was measured for 3D tissues 700 μm in diameter cultured for 3 days after treatment with 20 ng/mL of IGF.

FIG. 19A to FIG. 19D illustrates the metabolic activity (in fluorescence intensity) of sheep muscle spheroids of three sizes (300 μm, 500 μm, 700 μm in diameter) cultured in microchip for 3 days and 5 days following recombinant growth factor treatment with fibroblast growth factor (FGF), hepatocyte growth factor (HGF), and insulin-like growth factor (IGF). To monitor metabolic activity on-chip, 10 μM of Image-iT™ Red Hypoxia Reagent (Invitrogen, Austria) was prepared in respective cell growth medium. Culture medium was gently removed from reservoirs, and 200 μl of the 10 μM hypoxia reagent was applied. The chip was incubated for 1 hour at 37° C. and 5% CO2 in a live-cell incubator (Pecon, Germany) and imaged using TRITC filter (ex 530 nm, em 645 nm) by IX83 live-cell microscope (Olympus, Germany).

### Example 5—Spheroid Expansion Following Recombinant Growth Factor Treatment

FIG. 20A to FIG. 20B illustrates spheroid diameter of cells cultured in wells measuring 900 μm in diameter for 12 days following growth factor treatment with fibroblast growth factor (FGF), hepatocyte growth factor (HGF), and insulin-like growth factor (IGF). More specifically, FIG. 20A depicts spheroid diameter in and FIG. 20B depicts spheroid diameter as a percentage of size increase as compared to control. As depicted, the spheroid diameter is largest for spheroids treated with recombinant growth factors, with the largest difference occurring for IGF treated spheroids 6 days after treatment.

FIG. 21A to FIG. 21B depicts spheroid diameter of cells cultured in wells measuring 300 μm in diameter for 12 days following growth factor treatment with fibroblast growth factor (FGF), hepatocyte growth factor (HGF), and insulin-like growth factor (IGF). More specifically, FIG. 21A depicts spheroid diameter in and FIG. 21B depicts spheroid diameter as a percentage of size increase as compared to control. As depicted, the spheroid diameter is largest for spheroids treated with recombinant growth factors, with the largest difference occurring for HGF treated spheroids 3 days after treatment.

1.-50. (anceled)

51. A method of acquiring spheroids for a cultivated meat product comprising:
(a) acquiring muscle cells or muscle cell precursors from a non-human animal source;
(b) expanding the muscle cells or the muscle cell precursors from the non-human animal source in presence of an effective concentration of insulin-like growth factor (IGF) in a culture vessel, on a plate, or on a microfluidics chip to facilitate formation of muscle spheroids or organoids;
(c) harvesting the muscle spheroids or organoids when an average diameter of the muscle spheroids or organoids is about 100 micrometers or greater; and
(d) initiating further propagation in adherent or suspension cultures.

52. The method of claim 51, further comprising seeding a bioreactor with the adherent or suspension cultures for the cultivated meat product.

53. The method of claim 51, wherein acquiring the muscle cells or the muscle cell precursors from the non-human animal source comprises acquiring cells from a tissue biopsy, an immortalized cell line, blood, stem cells, precursor cells, embryonic cells, bone marrow, or any combination thereof.

54. The method of claim 51, further comprising screening the muscle cells, the muscle cells precursors, or the muscle spheroids or organoids for metabolic activity.

55. The method of claim 51, wherein the harvesting of the muscle spheroids or organoids occurs when the average diameter of the spheroids are from about 100 micrometers to about 1000 micrometers.

56. The method of claim 51, wherein the muscle spheroids or organoids comprise a single cell type.

57. The method of claim 51, wherein the muscle spheroids or organoids comprise a mixture of two or more cell types.

58. The method of claim 51, wherein the muscle spheroids or organoids further comprise embryonic stem cells, induced pluripotent stem cells, satellite cells, mesenchymal stem cells, and/or hematopoietic stem cells.

59. The method of claim 51, wherein the muscle spheroids or organoids comprise scaffolding.

60. The method of claim 51, wherein the muscle spheroids or organoids do not comprise scaffolding.

61. The method of claim 51, wherein the non-human animal is selected from the group consisting of: a cow, a pig, a chicken, a fish, a sheep, a bison, a duck, a goose, an elk, a deer, a Berkshire pig, a Kurobuta pig, an Iberian pig, and an ostrich.

62. The method of claim 51, wherein the muscle spheroids or organoids are cultured in a heterologous extracellular matrix.

63. The method of claim 51, wherein the muscle spheroids or organoids are cultured in a heterologous extracellular matrix comprising from about 5% to about 15% or from about 6% to about 14% gelatinous protein mixture from heterologous cells.

64. The method of claim 51, further comprising expanding the muscle cells or the muscle cell precursors from the non-human animal source by treating the muscle spheroids or organoids with fibroblast growth factor (FGF), hepatocyte growth factor (HGF), or a combination thereof.

65. The method of claim 51, wherein the muscle spheroids or organoids are treated with about 1 to about 50 ng/mL insulin-like growth factor (IGF).

66. The method of claim 51, wherein harvesting the muscle spheroids or organoids is performed at least 2 days after expanding the muscles cells or the muscle precursors from the non-human animal source in the presence of an effective concentration of insulin-like growth factor (IGF).

67. The method of claim 51, wherein harvesting the muscle spheroids or organoids is performed up to 5 days after expanding the muscles cells or the muscle precursors from the non-human animal source in the presence of an effective concentration of insulin-like growth factor (IGF).

68. The method of claim 51, wherein the culture vessel, the plate or the microfluidics chip comprise one or more recesses possessing an inverted dome shape and a diameter from about 100 micrometers to about 1,000 micrometers.

69. The method of claim 51, wherein the cultivated meat product comprises a second type of spheroid or organoid that is different from the muscle spheroids or organoids.

70. The method of claim 69, wherein the second type of spheroid or organoid comprises cells selected from a group consisting of muscle cell precursors, connective tissue cell precursors, fat cell precursors, chondrocyte precursors, blood cell precursors, and combinations thereof.
